SEQUENCE LISTING (25 sequences)

SEQ ID NO: 1 - DNA, 21 bp, Artificial Sequence
  Description of Artificial Sequence Primer
ggtaagggcctggagcaggaa

SEQ ID NO: 2 - DNA, 21 bp, Artificial Sequence
  Description of Artificial Sequence Primer
gcctcaacgtacccctgtctc

SEQ ID NO: 3 - DNA, 21 bp, Artificial Sequence
  Description of Artificial Sequence Primer
accgggcacctgtactcctca

SEQ ID NO: 4 - DNA, 22 bp, Artificial Sequence
  Description of Artificial Sequence Primer
gcatgagctaaggcacccagac

SEQ ID NO: 5 - DNA, 21 bp, Artificial Sequence
  Description of Artificial Sequence Primer
ccctcagcctcgtcacctcac

SEQ ID NO: 6 - DNA, 20 bp, Artificial Sequence
  Description of Artificial Sequence Primer
cacgtgcagggcacctagat

SEQ ID NO: 7 - DNA, 22 bp, Artificial Sequence
  Description of Artificial Sequence Primer
ccagaaggctttgcaggcttca

SEQ ID NO: 8 - DNA, 22 bp, Artificial Sequence
  Description of Artificial Sequence Primer
actgagccctgggaggtaggta

SEQ ID NO: 9 - DNA, 17 bp, Artificial Sequence
  Description of Artificial Sequence Primer
cgcatctcccaccccca

SEQ ID NO: 10 - DNA, 22 bp, Artificial Sequence
  Description of Artificial Sequence Primer
gactgccttcgccaaccactcc

SEQ ID NO: 11 - DNA, 26 bp, Artificial Sequence
  Description of Artificial Sequence Primer
gactcagcctcgtcacctcaccacag

SEQ ID NO: 12 - DNA, 29 bp, Artificial Sequence
  Description of Artificial Sequence Primer
actgactgactcggcctcctcggtcaccc

SEQ ID NO: 13 - DNA, 33 bp, Artificial Sequence
  Description of Artificial Sequence Primer
ctgactgactgggcctcctgctcatgatcctac

SEQ ID NO: 14 - DNA, 37 bp, Artificial Sequence
  Description of Artificial Sequence Primer
ctgactgactgactctcttgttgaccaggctggagtg

SEQ ID NO: 15 - DNA, 41 bp, Artificial Sequence
  Description of Artificial Sequence Primer
tgactgactgactgactctggatgagctgctaactgagcac

SEQ ID NO: 16 - DNA, 46 bp, Artificial Sequence
  Description of Artificial Sequence Primer
ctgactgactgactgactgactcgccaacgctgggctgcacgctac

SEQ ID NO: 17 - DNA, 25 bp, Artificial Sequence
  Description of Artificial Sequence Primer
tgactgcccgaaactcaggatctgg

SEQ ID NO: 18 - DNA, 38 bp, Artificial Sequence
  Description of Artificial Sequence Primer
ctgactgactgactagaacaggtcagccaccactatgc

SEQ ID NO: 19 - DNA, 25 bp, Artificial Sequence
  Description of Artificial Sequence Primer
tgactgcccaaaactcaggatctgg

SEQ ID NO: 20 - DNA, 36 bp, Artificial Sequence
  Description of Artificial Sequence Primer
ccggggtggttggcgaaggcagtcccctgtgctgcc

SEQ ID NO: 21 - DNA, 36 bp, Artificial Sequence
  Description of Artificial Sequence Primer
ccggagtggttggcgaaggcagtcccctgtgctgcc

SEQ ID NO: 22 - DNA, 23 bp, Artificial Sequence
  Description of Artificial Sequence Primer
aggtgactgccttcgccaaccac

SEQ ID NO: 23 - DNA, 24 bp, Artificial Sequence
  Description of Artificial Sequence Primer
aggtgactgccttcgccaaccact

SEQ ID NO: 24 - DNA, 23 bp, Artificial Sequence
  Description of Artificial Sequence Primer
aggtgactgccttcgccaaccac

SEQ ID NO: 25 - DNA, 23 bp, Artificial Sequence
  Description of Artificial Sequence Primer
aggtgactgccttcgccaaccac

(19) United States
(12) Patent Application Publication
(10) Pub. No.: US 2004/0091909 A1
(43) Pub. Date: May 13, 2004

(54) High throughput cytochrome P450 genotyping

(76) Inventor: Doug Hui Huang, Aliso Viejo, CA (US)

Correspondence Address: FOLEY & LARDNER, P.O. BOX 80278, SAN DIEGO, CA, 92138-0278, US

(21) Appl. No.: 10/615,497

(22) Filed: Jul. 7, 2003

Related U.S. Application Data
(60) Provisional application No. 60/393,967, filed on Jul. 5, 2002.
(60) Provisional application No. 60/396,618, filed on Jul. 16, 2002.

(51) Int. Cl. C12Q 1/68; C12P 19/34

(52) U.S. Cl. 435/6; 435/91.2

(57) ABSTRACT

Methods that provide for the rapid and simultaneous screening of large numbers of samples for several polymorphisms of a cytochrome P450 enzyme are disclosed. The assays can be used to rapidly determine if polymorphisms in a gene encoding a cytochrome P450 enzyme are present in the genome of an individual. An exemplary P450 enzyme to which the invention is applied is CYP2D6, which metabolizes many different drugs and other chemicals. Genetic polymorphisms in CYP2D6 and other P450 enzymes result in a range of phenotypes, from poor metabolizers to ultraextensive metabolizers, that have different abilities to metabolize chemicals including drugs. Identifying which polymorphisms are present in an individual patient allows one to tailor a drug therapy strategy that best fits that specific patient. The assays are thus useful in the field of pharmacogenetics.

### FIELD OF THE INVENTION

The invention relates to the field of pharmacogenetics, with particular regard to methods and compositions for determining the presence and sequence of variant alleles of genes encoding cytochrome enzymes involved in drug metabolism.

### BACKGROUND OF THE INVENTION

The following description of the background of the invention is provided simply as an aid in understanding the invention and is not admitted to describe or constitute prior art to the invention.

Pharmacogenetics

Different individuals, including patients being treated with therapeutic drugs, have differing responses to specific drugs and other chemicals. Some patients require a higher dose of a drug to achieve a therapeutic effect, or are more easily overdosed, or have a higher than average susceptibility specific drugs and other chemicals.

Genetic differences (polymorphisms) in genes encoding drug-metabolizing enzymes can be responsible for interindividual differences in drug response. In a population of individuals, polymorphisms in a gene encoding an enzyme that degrades drugs results in phenotypically-distinct subpopulations. By “phenotypically-distinct”, it is meant that the subpopulations differ in their ability to perform biotransformations of particular drugs. Thus, the genetic polymorphisms have phenotypic distinctions that may impact the selection of drugs to be used for a given individual patient, as each patient's genome contains a different set of polymorphisms.

For example, a drug that is safe when administered to most individuals may cause toxic side-effects in an individual exhibiting a genetic difference in an enzyme required for detoxification of the drug that results in reduced or enhanced metabolism of the drug relative to the average person. Conversely, a drug that is effective in most humans may be ineffective in a particular subpopulation because of lack of an enzyme required for conversion of the (pro) drug to a metabolically active form. Accordingly, it is important to identify individuals who have alterations in one or more drug-metabolizing enzymes, so that drugs known or suspected of being metabolized thereby are not used, or used only with special precautions (e.g., reduced dosage, close monitoring).

In pharmacogenetic studies, the genotype of polymorphic alleles encoding one or more drug-metabolizing enzymes is determined and linked to an individual's drug metabolism phenotype. Determination of these genetic polymorphisms may be of clinical value in predicting adverse or inadequate response to certain therapeutic agents and in predicting increased risk of environmental or occupational exposure-linked disease. For reviews,'see Weber et al., Pharmacogenetic Testing, Encyclopedia of Analytical Chemistry, Robert A. Myers, John Wiley & Sons Ltd., Chichester; Schmitz et al., Pharmacogenomics: implications for laboratory medicine, Clinica Chimica Acta 308:43-53, 2001; Linder et al., Pharmacogenetics: a laboratory tool for optimizing therapeutic efficiency, Clinical Chemistry 43:2, 254-266, 1997; Kalow, Pharmacogenetics in Biological Perspective, The American Society for Pharmacology and Experimental Therapeutics, 49:0369-0379, 1997. The goal of pharmacogenetics is to examine the genome of an individual patient and design a drug treatment strategy tailored to that patient's particular drug metabolism profile. Assays and other methods by which drug-metabolizing polymorphisms in an individual's genome are determined thus have utility in the field of pharmacogenetics. Preferably, such assays are accurate (e.g., few false positives or negatives) and performed quickly.

Cytochrome P450 Enzymes and Drug Metabolism

One group of drug-metabolizing enzymes that can cause variability in individual drug responses is the family of cytochrome P450 enzymes (Lu, Drug Metabolism And Disposition, 26:12, 1217-1222, 1998; van der Weide et al., Cytochrome P450 enzyme system: genetic polymorphisms and impact on clinical pharmacology, Ann Clin Biochem 36:722-729, 1999; Crespi et al., The use of heterologously expressed drug metabolizing enzymes—state of the art and prospects for the future, Pharmacology and Therapeutics 84:121-131, 1999). Cytochrome p450 enzymes are often designated by the letters CYP followed by a set of letters and numbers that distinguish enzyme isoforms. Understanding CYP enzyme interactions might allow prescribers the ability to better anticipate and manage each patient's response to a drug regimen.

The cytochrome P450 family of enzymes is primarily responsible for the metabolism of xenobiotics such as drugs, carcinogens and environmental chemicals, as well as several classes of endobiotics such as steroids and prostaglandins. Polymorphisms of cytochrome P450 enzymes result in phenotypically-distinct subpopulations that differ in their ability to perform biotransformations of particular drugs and other chemical compounds. The genetic polymorphisms present in an individual's genome may thus impact the selection and dosages of drugs to be used for that specific individual. For example, a higher level of activity of a cytochrome P450 enzyme that metabolizes a drug, measured relative to an average individual, may result in a lower level of that drug in the body. Accordingly, an individual having a high level of cytochrome P450 activity might have to be given a higher dosage of the drug in order to achieve an effective level thereof.

In addition to the direct effects that cytochrome P450 polymorphisms may have on the metabolism of drugs, drug interactions involving cytochrome P450 are also known. Differences in the levels of activity of cytochrome P450 enzymes due to genetic differences between individuals may thus influence choices of drug and dosage choices in order to avoid undesirable drug interactions. Drug interactions may result from either the inhibition or induction of a cytochrome P450 enzyme. Enzyme inhibition generally involves competition with another drug for enzyme binding sites and usually begins with the first dose of the inhibitor; the duration of inhibition varies with each respective drug. Enzyme induction occurs when one drug stimulates production of more enzymatic metabolism capacity.

Terfenadine is an infamous example of a drug interaction involving cytochrome P450 enzymes. Terfenadine is a prodrug that is normally metabolized in the body in its active form, Fexofenadine. This metabolic step normally occurs very quickly, so that the level of Terfenadine in the body is low. However, Terfenadine is metabolized by cytochrome P450 CYP3A, which is inhibited by the antifungal agents Ketoconazole and Itroconazole, as well as the antibiotic Erythromycin. Thus, treatment with Ketoconazole, Itroconazole or Erythromycin slows the metabolism of Terfenadine to Fexofenadine, and the level of Terfenadine in the body thus increases. At high levels, Terfenadine can cause abnormal beating of the heart (arrythmia) that can lead to death. An individual having a reduced level of the cytochrome P450 CYP3A enzyme would be more susceptible to this potentially fatal drug interaction.

Fluoxetine (Prozac) is an example of a drug that can have interactions with another drug due to different responses of different members of the cytochrome P450 family of enzymes. Fluoxetine is metabolized by a first cytochrome, CYP2D6, but inhibits the activity of a second cytochrome, CYP3A. The level of Fluoxetine thus influences the level of drugs that are metabolized by CYP3A, including Lovastatin. A CYP2D6 polymorphism that results in a poor metabolizer phenotype (PM) might have a relatively high level of Fluoxetine and, as a result, a higher level of unmetabolized Lovastatin. Thus, a patient having a CYP2D6 PM phenotype should be given a lower dose of Lovastatin in order to avoid any side effects associated with high levels of Lovastatin.

CYP2D6 and Assays Thereof

Cytochrome P4502D6 (CYP2D6), also known as debrisoquine hydroxylase, is one polymorphic P450 in the human population of particular interest. A poor metabolizer (PM) phenotype, that segregates as an autosomal recessive trait and which occurs with an incidence between 5 and 10% in the white population of North America and Europe, has been identified. Poor metabolizers (PM) exhibit negligible amounts of cytochrome P4502D6 (Gonzalez et al., Characterization of the common genetic defect in humans deficient in debrisquine metabolism, Nature 331:442-446, 1988). A poorly metabolizing individual may be better served by lower doses of various drugs.

In addition to influencing a patient's drug-metabolizing profile, genetic differences in CYP2D6 may be associated with increased risk of developing environmental and occupational based diseases (Gonzalez & Gelboin, J., Role of human cytochrome P-450s in risk assessment and susceptibility to environmentally based disease. Toxicology and Environmental Health 40:289-308, 1993). Individuals having a defect in a cytochrome P450 are often susceptible to cancers from environmental chemicals due to inability to detoxify the chemicals (Gonzalez et al., Role of human cytochromes P450 in the metabolic activation of chemical carcinogens and toxins, Drug, Metab. Rev. 26, 165-183; Gonzalez, The role of carcinogen-metabolizing enzyme polymorphisms in cancer susceptibility, Reproduct. Toxicol. 11, 397-412).

Determining the level of CYP2D6 activity in an individual thus has several beneficial utilities. Several ways of assessing levels of CYP2D6 activity are known and fall into the general categories of bioassays, immunoassays, cell culture assays and pharmacogenetic assays.

Thus, one way in which patient CYP2D6 profiles are assessed uses a bioassay after a probe drug administration. For example, a poor drug metabolizer with a CYP2D6 defect is identified by administering one a probe drug (e.g., debrisoquine, sparteine or dextromethorphan), then testing urine for the ratio of unmodified to modified drug. Poor metabolizers (PM) exhibit physiologic accumulation of unmodified drug and have a high metabolic ratio of probe drug to metabolite. See, e.g., Gonzalez et al., Clin. Pharmacokin. 26:59-70, 1994.

Another method for assessing CYP2D6 activity is by immunoassays using antibodies and other agents that bind to P4502D6 proteins (see, e.g., U.S. Pat. No. 6,060,253 to Gelboin et al., Agents that bind to and inhibit human cytochrome P450 2D6, issued May 9, 2000).

It is also known to culture hepatocytes to examine the activity of CYP2D6 and other P450s (LeCluyse et al., Human hepatocyte culture systems for the in vitro evaluation of cytochrome P450 expression and regulation, European Journal of Pharmaceutical Sciences 13 343-368, 2001).

Assays that make use of DNA sequences to identify known polymorphisms may also be used to detect and identify polymorphisms. Examples of genetic assays of CYP2D6 are described by Schur et al., Genotyping of cytochrome P450 2D6*3 and *4 mutations using conventional PCR, Clinca Chimica Acta 308, 25-31, 2001; Hersberger et al., Rapid detection of the CYP2D6*3, CYP2D6*4, and CYP2D6*6 alleles by tetra-primer PCR and of the CYP2D6*5 allele by multiplex long PCR, Clinical Chemistry 46:8, 1072-1077, 2000; Meyer, Primers targeted to CYP2D6 gene for detecting poor metabolizers of drugs, 1997, U.S. Pat. No. 5,648,482; Sinnett et al., Detection of CYP1A1, CYP3A4, CYP2D6 and NAT2 variants by PCR-allele-specific oligonucleotide (ASO) assay, 2001, U.S. Pat. No. 6,183,963 B1; and Linder et al., Pharmacogenetics: a laboratory tool for optimizing therapeutic efficiency, Clinical Chemistry 43:254-266, 1997). Genetic assays for other P450s are known; see, e.g., Kamataki et al, CYP2A6 Gene Judgment Methods, WO 00/66775, 2000; and Wolf et al., Genetic Assay, U.S. Pat. No. 5,981,174, Nov. 9, 1999.

### SUMMARY OF THE INVENTION

The present invention is drawn to methods and compositions for the rapid and simultaneous screening of samples for a plurality of cytochrome P450 polymorphisms. The sample can be a biological sample, such as a sample from a subject. The invention can be used to rapidly determine which of a plurality of P450 polymorphisms are present in the genome of a subject. In accordance with the method, one may determine wildtype and several different cytochrome P450 polymorphisms in a single reaction mixture of a single sample. Thus, a plurality of polymorphisms may be simultaneously assayed for several different P450 polymorphisms and wildtype in a single cycle (batch run) of the assay.

In a first aspect, the invention provides methods of testing for the presence of one or more polymorphisms of a cytochrome P450 gene, in one or more samples comprising nucleic acid, the nucleic acid having a nucleotide sequence that modulates the expression and/or encodes the cytochrome P450 gene, by generating a labeled nucleic acid that provides a means of identifying a particular polymorphism and distinguishing that polymorphism from other polymorphisms that might be present in the same gene. The particular polymorphism may be identified, for example, by determining both the length of the labeled nucleic acid and the identity of a distinctively labeled nucleotide incorporated at an end of the nucleic acid.

In preferred embodiments, these methods comprise one or more of the following steps: (a) preparing a reaction mixture that contains (i) an amount of a cytochrome P450 nucleic acid sufficient for primer extension, (ii) a nucleic acid polymerase, (iii) one or more extension primers, wherein the extension primers comprise nucleotide sequences that terminate at positions located one nucleotide 5 from the positions of the preselected polymorphism(s) of interest, and (iv) a set of distinctively labeled dideoxynulceotide triphosphates, or ddNTPs; (b) incubating the reaction mixture under conditions such that extension primers that hybridize to the nucleic acids are distinctively labeled by addition of one of the ddNTPs comprising a label to the 5′-end of the detection primer, in order to generate a set of distinctively labeled oligonucleotides; and (c) detecting a set of distinctive signals from the set of distinctively labeled oligonucleotides. The presence of a specific polymorphism can be identified by the presence of a distinctive signal at a position in the sequence of the extended nucleic acid.

The term “biological sample” as used herein refers to a sample obtained from a biological source, e.g., an organism, cell culture, tissue sample, etc. A biological sample can, by way of non-limiting example, consist of or comprise blood, sera, urine, feces, epidermal. sample, skin sample, cheek swab, sperm, amniotic fluid, cultured cells, bone marrow sample and/or chorionic villi.

The term “subject” as used herein refers to any eukaryotic organism. Preferred subjects are fungi, invertebrates, insects, arachnids, fish, amphibians, reptiles, birds, marsupials and mammals. A mammal can be a cat, dog, cow, pig, horse, ox, elephant, simian or human. Most preferred subjects are humans. A subject can be a patient, which refers to a human presenting to a medical provider for diagnosis or treatment of a disease. Methods to study the role of cytochrome P450 enzymes in metabolism in animals are known (see, e.g., Chauret et al., In Vitro Comparison of P450-Mediated Metabolic Activities in Human, Dog, Cat, and Horse, Drug Metabolism and Disposition 25:1130-1136, 1997). The term “animals” includes metamorphic and prenatal forms of animals.

In the disclosure, a “plurality of samples” refers to at least two. Preferably, a plurality refers to a relatively large number of samples. A plurality of samples is from about 5 to about 500 samples, preferably about 25 to about 200 samples, most preferably from about 50 to about 200 samples. Samples that are processed in a single batch run of the method of the invention are usually prepared in plates having 24, 48, 96, 144, or 192 wells. The term “samples” includes samples per se as well as controls, standards, etc. that are included in a batch run.

A preselected cytochrome P450 gene is a cytochrome P450 gene or protein that has been selected for testing according to the invention. By way of non-limiting example, the preselected cytochrome P450 gene can be in one or more of CYP1A1, CYP1 A2, CYP2A6, CYP2B6, CYP2C9, CYP2Cl8, CYP2Cl9, CYP2D6, CYP2E1, CYP3A4 or CYP3A5. As is explained in more detail below, CYP2D6 is a representative cytochrome P450 gene used in the illustrative Examples of the disclosure.

“One or more preselected cytochrome P450 polymorphisms” refers one or more polymorphisms in a preselected cytochrome P450 gene that have been selected for testing according to the invention. For example, in the case of CYP2D6, the mutation may be any one or more disclosed in Table 1 including CYP2D6*2, CYP2D6*3, CYP2D6*4, CYP2D6*5, CYP2D6*6, CYP2D6*7, CYP2D6*8, CYP2D6*9, CYP2D6*10, CYP2D6*11, CYP2D6*12, CYP2D6*13, CYP2D6*14, CYP2D6*15, CYP2D6*16, CYP2D6*17, CYP2D6*1×2, CYP2D6*2×2, CYP2D6*4×2, and CYP2D6*Nx2 (detecting any of * 1×2, *2×2 or *4×2).

The assays can be used to rapidly determine if polymorphisms in a gene encoding a cytochrome P450 enzyme are present in a sample comprising nucleic acid. By “rapid” it is meant that the length of time that is taken to carry out a single batch run of the assay, from the moment a reaction mixture comprising nucleic acid is prepared to the moment a signal can be detected, is from about 1 second to about 10, 15 or 30 seconds, about 1, 5, 10 or 30 minute(s), or 1, 3, 5, 8, 24 or 48 hour(s). When samples are from individual subjects, the assays can be used to determine the cytochrome P450 genotype of each subject.

By “distinctively labeled”, it is meant that each type of member of a set is labeled with a distinct label that can be distinguished from the other labels. For example, in a set of distinctively labeled nucleotides (e.g., dideoxy NTPs, or ddNPTs), each type of “N” (nucleotide) is labeled with a label that can be distinguished from the other types of labels. Thus, for example, if four labels designated 1, 2, 3, and 4 are used to label the four types of ddNTPs, each ddATP molecule is labeled with label “*1”, each ddTTP molecule is labeled with label “*2”, each ddCTP molecule is labeled with label “*3”, and each ddGTP molecule is labeled with label “*4”. In some aspects of the invention, the distinctive label is a fluorescent label.

As used herein, “primer extension” refers to the enzymatic extension of the three-prime (3′) hydroxy group of an extension primer, which is an oligonucleotide X nucleotides long that is paired to a template nucleic acid (for an example of primer extension as applied to the detection of polymorphisms, see Fahy et al., Mutliplex fluorescence-based primer extension method for quantative mutation analysis of mitrochondrial DNA and its diagnostic application for Alzheimer's disease, Nucleic Acid Research 25:3102-3109, 1997). The extension reaction is catalyzed by a DNA polymerase. By “DNA Polymerase” it is meant a DNA polymerase, or a fragment thereof, that is capable of carrying out primer extension. Thus, a DNA polymerase can be an intact DNA polymerase, a mutant DNA polymerase, an active fragment from a DNA polyerase, such as the Klenow fragment of E. coli DNA polymerase, and a DNA polymerase from any species, including but not limited to thermophilies.

Extension of the 3′ end of the oligonucleotide generates an oligonucleotide having a length of at least (X+Y) nucleotides, where Y>1, having a sequence that is the reverse complement of the template nucleic acid. If one of the nucleotides in the added sequence Y is labeled, then the extended (X+Y) oligonucleotide is labeled.

An extension primer has a nucleotide sequence has a sequence that binds in a complementary fashion to a portion of a sequence of a nucleic acid that encodes or modulates the expression of the cytochrome P450, or to the complement of such a sequence. Preferred extension primers are of a length sufficient to provide specific binding to the sequence of interest. Such primers comprise an exact complement to the sequence of interest for 15 to 40 nucleotides in length, and preferably 20 to 30 nucleotides in length. The extension primer sequence has a 3′ terminus that pairs with a nucleotide base that is, in the sample nucleic acid to which the primer is hybridized, 5′ from the site of one or more bases in the sequence of interest that represent a polymorphism in a gene.

For example, in the following diagram of a primer extension reaction, four different ddNTPs, each distinctively labeled, are present in the reaction mixture as designated by dd(A* 1)TP, dd(T*2)TP, dd(C*3)TP and dd(G*4)TP, where * 1, *2, *3 and *4 represent different labels. In the diagram, the polymorphism in the nucleic acid being tested is indicated by an underlined nucleotide, and the extension primer sequence is italicized. Only one ddNTP, ddTTP, can be added to the 3′ end of the extension primer, because thymine (T) is the only base that pairs with adenosine (A). The addition of the dd(T*2)TP to the 3′ of the primer prevents any further primer extension because it is a dideoxy, chain-terminating ddNTP. Thus, the only primer that is 3′ extended is labeled with label *2. Detection of the signal from label *2 indicates that the A polymorphism is present in the sample.

| | |
|---|---|
| wildtype | 5′ CCGGGGTGGTTGGCGAAGGCAGTCCCCTGTGCTGCC -3′ |
| sample | 5′ CCGGAGTGGTTGGCGAAGGCAGTCCCCTGTGCTGCC -3′ |
| | \|\|\|\|\|\|\|\|\|\|\|\|\|\|\|\|\|\|\|\|\|\|\| |
| primer | 3′ CACCAACCGCTTCCGTCAGTGGA -5′ |
| labeled ddNTP | |
| dd(A*¹)TP | 3′ CACCAACCGCTTCCGTCAGTGGA -5′ |
| dd(T*²)TP | 3′ *^{2 T}CACCAACCGCTTCCGTCAGTGGA -5′ |
| dd(C*³)TP | 3′ CACCAACCGCTTCCGTCAGTGGA -5′ |
| dd(G*⁴)TP | 3′ CACCAACCGCTTCCGTCAGTGGA -5′ |

An amount of nucleic acid sufficient for primer extension can, but need not be, prepared by amplification via polymerase chain reaction (PCR) using PCR primers. As a non-limiting example, when the preselected cytochrome P450 gene is CYP2D6, appropriate PCR primers include, but are not limited to, those having sequences selected from the group consisting of SEQ ID NOS: 1 through 8. See Table 2.

For each reaction mixture, the amount of the nucleic acid sufficient for primer extension is determined by obtaining a sample comprising nucleic acid and determining the concentration of nucleic acid therein. One skilled in the art will be able to prepare such samples to a concentration and purity necessary to practice the invention, and to estimate the amount of a specific sample that should be added to a particular reaction mixture. A failure to detect a signal in the method of the invention signifies that, among other things, an inadequate amount of nucleic acid has been added to a reaction mixture. Those skilled in the art will be able to trouble-shoot failed batch runs and adjust the contents of the reaction mixtures and/or conditions of the run accordingly. Control samples can be included in the batch runs to confirm that appropriate amounts of nucleic acid are present.

One or more of steps (a), (b) or (c), or combinations thereof, are preferably performed automatically, typically using robotics, in order to provide for the processing of a large number of samples in a single batch run. Preferred forms of automation will provide for the preparation and separation of a plurality of labeled nucleic acids in small volumes. The term “small volumes” refers to volumes of liquids less than 2 ml, e.g., any volume from about 0.001 picoliters or about 0.001 μl, to any volume about 2 ml, 500 μl, 200 μl, 100 μl, 10 μl, 1 μl, 0.1 μl, 0.01 μl, or 0.001 μl.

The set of distinctively labeled oligonucleotides can be separated from each other so that each is mobilized in a manner that relates to each of their specific positions in the respective nucleotide sequence, and the detection of the distinctive signals generated from the distinctively labeled oligonucleotides occurs during or after the mobilization (i.e., during step(c), or after step (b) but before step (c)). Members of the set of distinctively labeled oligonucleotides can be separated from each other so that each is mobilized by electrophoresis. A preferred form of electrophoresis is capillary electrophoresis, or any form of electrophoresis that allows for the separation of a plurality of labeled nucleic acids in small volumes by automated or semi-automated methods and devices.

The cytochrome P450 polymorphisms can be of any type, including, but are not limited to, deletions, inversions, insertions, translocations, polymorphisms resulting in aberrant RNA splicing, single nucleotide polymorphisms, and combinations thereof In the Examples, for purposes of illustration, the preselected cytochrome P450 is CYP2D6 and the polymorphisms are thus CYP2D6 polymorphisms. Representative CYP2D6 wildtype and polymorphisms include those in Table 1. By way of non-limiting example, the preselected cytochrome P450 polymorphism can be one or more of CYP2D6*3, CYP2D6*4, CYP2D6*5, CYP2D6*6, CYP2D6*7, CYP2D6*8, CYP2D6*10, CYP2D6*17, and CYP2D6*N×2. Extension primers for many of these polymorphisms are described herein and have one of the sequences of SEQ ID NOS: 9 through 19. See Table 3.

In an alternative approach, the method provides for detection of one or more P450 2D6 wildtype or mutations. The method comprises incubating a reaction comprising: (i) an amount of nucleic acid obtained from said sample sufficient for primer extension, wherein said nucleic acid comprises said P450 2D6 gene sequence, (ii) a nucleic acid polymerase, (iii) at least one extension primer selected from the group consisting of SEQ ID NOs 9 to 19, and (iv)a set of distinctively labeled ddNTPs, under conditions such that said at least one extension primer is distinctively labeled by addition of one of said ddNTPs comprising a label to the 5′-end of said at least one detection primer, to generate at least one labeled nucleic acid corresponding to at least one of said preselected polymorphisms; and relating the labeled nucleic acid to the identity of said polymorphism in said sample.

In one aspect of the invention, polymorphisms assayed according to the invention are preferably associated with phenotype that effects the metabolism of an undesirable xenobiotic, such as a toxin, or a therapeutic xenobiotic, such as a drug or prodrug. The phenotypes include, but are not limited to, having a reduced rate or degree of metabolism of one or more xenobiotics or endobiotics, having an increased rate or degree of metabolism of one or more xenobiotics or endobiotics, having a decreased or increased specificity for one or more xenobiotics or endobiotics, and combinations of any of these. In certain embodiments, the identification of a polymorphism is used to select the administration or dose of a drug to a subject, preferably a patient.

The xenobiotic can be an undesirable compound, such as a toxin, a carcinogen or a narcotic, or a metabolic precursor thereof In this aspect of the invention, assays are carried out on subjects having, or suspected of having, a genetic predisposition to suffer from a toxin, to develop tumors, or abuse of a narcotic, respectively. The assays of the invention are used to determine what prophylactic procedures or treatments should be used by or applied to a given subject. That is, for example, a subject having a cytochrome P450 polymorphism associated with an increased risk of developing cancer can be treated with anticancer agents and procedures, including surgeries, or encouraged to avoid carcinogens and their metabolic precursors that are metabolized by the preselected cytochrome P450 enzyme.

The xenobiotic can be a desirable compound, such as a therapeutic drug or a metabolic precursor thereof Metabolic precursors of drugs include prodrugs, i.e., agents that are not active when administrated to a subject but which are metabolized to an active compound within the body of the subject. In the case of CYP2D6, therapeutic drugs of particular interest include cardioactive drugs and psychoactive drugs. Cardioactive drugs include by way of non-limiting example beta-blockers, including but not limited to bufuralol, propranolol, metoprolol, and timolol; and antiarrhythmics, including but not limited to sparteine, encainide, flecainide, mexiletine and N-propylamine. Psychoactive drugs include by way of non-limiting example neuroleptics, including but not limited to codeine, flupherazine, halopidol, levopromazin, thioridazine; selective serotonin reuptake inhibitors, including but not limited to venlafaxine, citalopram, fluoxetine and paroxetine; anxiolytics including but not limited to diazepam, nitrazepam, and clonazepam; antipsychotics, including but not limited to clozapine and haloperidol; tricyclic antidepressants, including but not limited to imipramine, clomipramine, desipramine, nortriptyline and amitryptyline; anticonvulsants; analgesics; and narcotics including but not limited to codeine, amphetamine and cocaine.

In a related aspect, the subject has a disease or disorder that may be treated by a therapeutic drug that is, or has a metabolic precursor that is, metabolized by the preselected cytochrome P450 enzyme. Diseases and disorders to which the invention can be applied include, by way of non-limiting example, the following.

Diseases and disorders that involve the respiratory system, such as cystic fibrosis, lung cancer and tumors, asthma, pathogenic infections, allergy-related diseases and disorders, such as asthma; allergic bronchopulmonary aspergillosis; hypersensitivity pneumonia, eosinophilic pneumonia; emphysema; bronchitis; allergic bronchitis bronchiectasis; cystic fibrosis; hypersensitivity pneumotitis; occupational asthma; sarcoid, reactive airway disease syndrome, interstitial lung disease, hyper-eosinophilic syndrome, parasitic lung disease and lung cancer, asthma, adult respiratory distress syndrome, and the like Legrand-Andreoletti et al., Cytochrome P450 CYP2D6 gene polymorphism and lung cancer susceptibility in Caucasians, Pharmacogenetics 8:7-14, 1998; Guidice et al., Evidence for CYP2D6 expression in human lung, Biochem Biophys Res Commun 241:79-85, 1997.

Diseases and disorders of the digestive system, such as those of the gastrointestinal tract, including cancers, tumors, pathogenic infections, colitis; ulcerative colitis, diverticulitis, Crohn's disease, gastroenteritis, inflammatory bowel disease, bowel surgery ulceration of the duodenum, a mucosal villous disease including but not limited to coeliac disease, past infective villous atrophy and short gut syndromes, pancreatitis, disorders relating to gastroinstestinal hormones, Crohn's disease, and the like;

Diseases and disorders of the skeletal system, such as spinal muscular atrophy, rheumatoid arthritis, osteoarthritis, osteoporosis, multiple myeloma-related bone disorder, cortical-striatal-spinal degeneration, and the like;

Autoimmune diseases, such as Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis amyotrophic lateral sclerosis, multiple sclerosis, autoimmune gastritis, systemic lupus erythematosus, autoimmune hemolytic anemia, autoimmune neutropenia, systemic lupus erythematosus, graft vs. host disease, bone marrow engraftment, some cases of Type I diabetes, and the like (Oliver et al., Use of Single Nucleotide Polymorphisms (SNP) and Real-Time Polymerase Chain Reaction for Bone Marrow Engraftment Analysis, Journal of Molecular Diagnostics 2:202-208, 2000; Sabbagh et al., Genetic analysis of the cytochrome P450 CYP2D6 polymorphism in patients with systemic lupus erythematosus, Pharmacogenetics 8:191-4, 1998);

Neurological diseases and disorders, such as depression, bipolar disorder, schizophrenia, Alzheimer's disease, Parkinson's disease, familial tremors, Gilles de la Tourette syndrome, eating disorders, Lewy-body dementia, chronic pain and the like (Mogil, The genetic mediation of individual differences in sensitivity to pain and its inhibition, Proc. Natl. Acad. Sci. 96:7744-7751, 1999).

Pathological diseases and resultant disorders such as bacterial infections such as infection by Escherichia, Shigella, Salmonella; sepsis, septic shock, and bacteremia; infections by a virus such as mHV, adenovirus, smallpox virus, hepatovirus, and the like; and AIDS-related encephalitis, HIV-related encephalitis, chronic active hepatitis, and the like;

Proliferative disease and disorders, such as acute lymphoblastic leukemia, acute myelogenous leukemia, chronic myelogenous leukemia, metastatic melanoma, Kaposi's sarcoma, multiple myeloma, breast cancer, anal cancer, vulvar cancer, and the like (Krajinovic et al., Susceptibility to Childhood Acute Lymphoblastic Leukemia: Influence of CYP1A1, CYP2D6, GSTM1, and GSTT1 Genetic Polymorphisms, The American Society of Hematology 93:1496-1501, 1999; Chen et al., CYP2D6 Genotype and the Incidence of Anal and Vulvar Cancer, Cancer Epidemiology, Biomarkers & Prevention 8:317-321, 1999; Dunning et al., A Systematic Review of Genetic Polymorphisms and Breast Cancer Risk, Cancer Epidemiology, Biomarkers & Prevention 8:843-854, 1999); and

Various diseases, disorders and traumas including, but not limited to, apoptosis mediated diseases, inflammation, cerebral ischemia, myocardial ischemia, aging, sarcoidosis, granulomatous colitis, scleroderma, degenerative diseases, necrotic diseases, alopecia, neurological damage due to stroke, diffuse cerebral cortical atrophy, Pick disease, mesolimbocortical dementia, thalamic degeneration, Huntington chorea, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, Hallervorden-Spatz disease, Meige syndrome, acanthocytic chorea, Friedreich ataxia, Holmes familial cortical cerebellar atrophy, Gerstmann-Straussler-Scheinker disease, progressive spinal muscular atrophy, progressive balbar palsy, primary lateral sclerosis, hereditary muscular atrophy, spastic paraplegia, glomeralonephritis, chronic thyroiditis, Grave's disease, thrombocytopenia, myasthenia gravis, psoriasis, peroneal muscular atrophy, hypertrophic interstitial polyneuropathy, heredopathia atactica polyneuritiformis, optic neuropathy, and ophthalmoplegia.

The results from the assays of the invention can be used to design a regimen of drug treatment that matches an individual subject's P450 polymorphisms. For example, a drug that is administered in a relatively toxic form, but which is quickly metabolized to a non-toxic form by the preselected cytochrome P450 enzyme of choice, is preferably not administered, or administered in lower doses, to a patient that has a poor metabolizer phenotype that is associated with a polymorphism in that cytochrome P450 gene. As another non-limiting example, the cytochrome P450 polymorphisms that are detected by the assays of the invention can be involved in causing or mediating undesirable drug-drug interactions. In these instances, a drug regimen can be prepared that avoids or minimizes the drug-drug interactions.

The invention thus provides a method of selecting a therapeutic drug or prodrug to treat a subject suffering from a disease or disorder comprising (a) obtaining a sample from the subject, wherein the sample comprises nucleic acid, the nucleic acid having at least one nucleotide sequence selected from the group consisting of (i) a nucleotide sequence that encodes a preselected cytochrome P450 protein, (ii) a nucleotide sequence that has the reverse complement of a nucleotide sequence that encodes the cytochrome P450 protein, and (iii) a nucleotide sequence that modulates the expression of (i) or (ii); (b) preparing a reaction mixture that contains (i) an amount of the nucleic acid sufficient for primer extension, (ii) a nucleic acid polymerase; (iii) one or more extension primers, wherein the extension primers comprise nucleotide sequences that terminate at positions located one nucleotide 5′ from the positions of the polymorphisms, and (iv) a set of distinctive labeled ddNTPs; (c) incubating the reaction mixture under conditions such that extension primers that hybridize to the nucleic acids are distinctively labeled by addition of one of the ddNTPs comprising a label to the 5′-end of the detection primer, in order to generate a set of distinctively labeled oligonucleotides; and (d) detecting a set of distinctive signals from the set of distinctively labeled oligonucleotides; wherein the presence or absence of the signal is related to the presence or absence of one or more allelic variants of the cytochrome P450 gene, and wherein the cytochrome P450 protein metabolizes the therapeutic drug or prodrug.

In a another aspect, the invention provides substantially purified nucleic acid extension primers that are selected from the group consisting of SEQ ID NOs. 9 to 19. By “substantially pure” a nucleic acid, or combination of nucleic acids, represents more than 50% of the nucleic acid in a sample. The nucleic acid sample may exist in solution or as a dry preparation.

The summary of the invention described above is non-limiting and other features and advantages of the invention will be apparent from the following detailed description of the invention, and from the claims.

### DETAILED DESCRIPTION OF THE INVENTION

The invention is drawn to pharmacogenetic assays. In particular, the invention provides methods and compositions for determining the presence and sequence of variant alleles of genes encoding cytochrome P450 enzymes involved in drug or toxin metabolism. More particularly, the invention is drawn to pharmacogenetic assays of human cytochrome P450 2D6 (CYP2D6).

Polymorphisms

In a normal diploid eukaryote, each gene has 2 loci, i.e., 1 gene copy at the same locus (position) on each of 2 matched chromosomes. Different versions of a gene can occur at any locus, and these versions are called alleles. Alleles include the wildtype (normal) allele and allelic variants.

By “allelic variant” it is meant a variation in a nucleotide sequence, such as a single nucleotide polymorphism (SNP) or any other variant nucleic acid sequence or structure (e.g., duplications, deletions, inversions, insertions, translocations, etc.) in a gene encoding a gene that alters the activity and/or expression of the gene. Allelic variants and/or over- or under-express the polypeptide encoded by the gene, and/or express proteins altered activities by virtue of having amino acid sequences that vary from wildtype sequences.

As used herein, expression refers to genetic expression as that term is used in the art, and thus encompasses alterations in the level of the protein encoded by a gene. Over-expression occurs when a variant gene is expressed at levels higher than those of the corresponding wildtype gene. Conversely, under-expression indicates that the variant gene is expressed at levels lower than the wildtype gene. An altered activity of a protein can be, by way of non-limiting example, a change in the rate or degree of a reaction catalyzed by an enzyme, an altered substrate specificity, and the like.

Often, more than one allelic variants exist and persist in a population of individuals. By “exist and persist” it is meant that the frequency of incidence of the rarer allele(s) is greater than can be explained by recurrent mutation alone (i.e., typically greater than 1%). However, the frequency of any variant allele may vary over time due to such factors as genetic drift and the like. When 2 or more variant alleles of a gene are present in a population, the gene or the protein it encodes is said to be polymorphic. As used herein, a “polymorphism” refers to a specific allelic variant of a gene or protein.

As is explained in more detail below, members of the cytochrome P450 family catalyze the metabolism of many xenobiotics and endobiotics. An endobiotic is a chemical compound that exists naturally in an individual; examples include proteins, steroids, etc. A xenobiotic is a chemical compound that does not naturally exist in an individual. Some xenobiotics, such as therapeutic drugs, have a beneficial effect when present in an individual. Others, such as toxins and carcinogens, have detrimental effects. Thus, polymorphisms in P450 enzymes and other metabolizing enzymes can be associated with marked differences in response to drug therapy and/or may also cause increased susceptibility to environmentally based diseases such as cancer. Differences in the metabolism of drugs can lead to severe toxicity or therapeutic failure by altering the relation between dose and blood concentration of the pharmacologically active drug.

Because a population of individuals can have major variations in the activities of enzymes that degrade drugs, screening tests can be undertaken under the supervision of a physician to select a preferred drug regimen for any specific individual. The preferred dosage of any such drug can be determined through trial-and-error tests (more accurately described as “trial-and-adjustment” tests). In this procedure, a patient is prescribed an initial dosage of a drug in order to establish certain baseline values to ensure that the patient is not a poor metabolizer of the drug, and to ensure that the patient does not suffer an adverse reaction to the drug. After the baseline test has been completed, the patient is also given a very low “starting” or a “best guess” dosage of the drug for a period such as one or two weeks. At the end of this trial period, the patient's response to the drug is evaluated. Based on the evaluation of the patient, the dosage of the drug can be adjusted for the next 1 or 2 week trial period.

Variability in the activity of a drug-metabolizing enzyme occurs due to the presence of one or more polymorphisms in the gene encoding the enzyme. In pharmacogenetic studies, the genotype of polymorphic alleles encoding one or more drug-metabolizing enzymes is determined and linked to an individual's drug metabolism phenotype. Determination of the genetic polymorphisms that are associated with different metabolic phenotypes has the clinical value of predicting adverse or inadequate response to certain therapeutic agents, and in predicting increased risk of environmental or occupational exposure-linked disease. Pharmacogenetics thus provides a rapid and accurate way of predicting the phenotype of an individual and quickly choosing a dosing regimen tailored to that specific phenotype.

Cytochrome P450 Enzymes

The family of enzymes known as “cytochrome P450” enzymes (since they absorb light in the 450 nanometer range), or as “cytochrome oxidase” enzymes (since they oxidize a wide range of compounds that do not naturally occur in circulating blood), encompasses a variety of enzymes, many of which are involved in xenobiotic metabolism, including by way of non-limiting example the metabolism of drugs, prodrugs and toxins. Directories and databases of P450s, and information regarding their substrates, are available on-line (Fabian et al., The Directory of P450-containing Systems in 1996, Nucleic Acids Research 25:274-277, 1997). In humans, at least about 200 different P450s are present (for a review, see Hasler et al., Human cytochromes P450, Molecular Aspects of Medicine 20:1-137, 1999). There are multiple forms of these P450s and each of the individual forms exhibit degrees of specificity towards individual compounds or sets of compounds. In some cases, a substrate, whether it is a drug or a carcinogen, is metabolized by more than one cytochrome P450 enzyme.

Members of the cytochrome P450 family are present in varying levels and their expression and activities are controlled by variables such as chemical environment, sex, developmental stage, nutrition and age. The cytochrome P450s are found at high concentrations in liver cells, and at lower concentrations in other organs and tissues such as the lungs (e.g., Fonne-Pfister et al., Xenobiotic and endobiotic inhibitors of cytochrome P-450db1 function, the target of the debrisoquine/sparteine type polymorphism, Biochem. Pharmacol. 37:3829-35, 1988). By oxidizing lipophilic compounds, which makes them more water-soluble, cytochrome oxidase enzymes help the body eliminate (via urine, or in aerosols exhaled out of the lungs) compounds that might otherwise act as toxins or accumulate to undesired levels.

In humans, several cytochrome P450 genes and enzymes encoded thereby have been identified as being involved in xenobiotic metabolism. These include CYP1A1, CYP1A2, CYP2A6, CYP2B6, CYP2C8, CYP2C9, CYP2C18, CYP2Cl9, CYP2D6, CYP2E1, CYP3A4, and CYP3A5 (Crespi et al., The use of heterologously expressed drug metabolizing enzymes—state of the art and prospects for the future, Pharm Ther 84:121-131, 1999). Table 4 herein provides a list of agents that have been reported to be metabolized by, inhibit, or induce specific cytochrome P450 isoforms. In many cases, allelic variants in the genes encoding cytochrome P450s have been identified. Of particular interest is the exemplary cytochrome P450-2D6 enzyme that is assayed according to the invention in the Examples.

Human Cytochrome P450 2D6 (CYP2D6)

A debrisoquin hydroxylase enzyme that fell within the cytochrome P-450 class of enzymes was initially referred as “cytochrome P450-DB”, where “DB” referred to debrisoquin. An oxygenase enzyme in liver tissues that degrades an entirely different drug, sparteine, was later characterized and named sparteine monooxygenase. It was later realized that debrisoquin hydroxylase and sparteine monooxygenase are the same enzyme. As the nomenclature of P450 enzymes evolved into a standardized system, the debrisoquin hydroxylase/sparteine monooxygenase enzyme came to be known as the cytochrome P450-2D6 enzyme. Thus, the same enzyme (Ec 1.14.14.1) has been referred to by different names: debrisoquin hydroxylase, cytochrome P450-DB, sparteine monooxygenase, cytochrome P450-2D6, CYPIID6 and CYP2D6. The term CYP2D6 is used herein.

About 25% of prescribed drugs are metabolized by CYP2D6. Cardiovascular drugs and drugs used for treatment of psychiatric disorders appear to be most directly effected by CYP2D6 polymorphisms. The list of therapeutically important compounds metabolized by CYP2D6 includes cardioactive drugs: beta-blockers (bufuralol, propranolol, metoprolol, timolol); antiarrhythmics (sparteine, encainide, flecainide, mexiletine, N-propylamine) (Woosley et al., Clinical implications of variable antiarrhythmic drug metabolism, Pharmacogenetics 2:2-11, 1992; and Birgersdotter et al., Steroselective genetically-determined interaction between chronic flecainide and quinidine in patients with arrhythmias, Brit J. Clin. Pharmacol. 33:275-280, 1992); psychoactive drugs including tricyclic antidepressants (imipramine, desipramine, nortriptyline, amitryptyline), antidepressants (venlafaxine a.k.a. Effexor, fluoxetine a.k.a. Prozac, Paroxetine (Paxil), and antipsychotics (clozapine and haloperidol) (Dahl & Bertilsson, Genetically variable metabolism of antidepressants and meuroleptic drugs in man, Pharmacogenetics 3:61-70, 1993; Fischer et al., The antipsychotic clozapine is metabolized by the polymorphic human microsomal and recombinant cytochrome P450 2D6, J. Pharmacol. Exp. Ther. 260:1355-1360, 1992; Stimer et al., Pharmacogenetics: a new diagnostic tool in the management of antidepressive drug therapy, Clinical Chimica Acta 308:33-41, 2001; Hiemke et al., Pharmacokinetics of selective serotonin reuptake inhibitors, Pharmacology & Therapeutics 85:11-28, 2000; and Eichelbaum & Gross, The genetic polymorphism of debrisoquine/sparteine metabolism—clinical aspects, Pharmac. Ther. 46:377-394, 1990); and opiod drugs and narcotics (codeine, amphetamine and cocaine).

The cDNA for human cytochrome CYP2D6 has been cloned and sequenced (Gonzalez et al., 1988). The genomic sequence of CYP2D6 is also known. CYP2D6 encompasses 9 exons spanning 4.66 kb at chromosomal locus 22ql3.1. These and other CYP2D6 sequences are available from databases such as GenBank (Accession numbers XM_{—}040063, XM_{—}040066, XM_{—}4040064, XM_{—}040062, XM_{—}040060, XM_{—}013013, and XM_{—}040065). The availability of these sequences and the advent of molecular genetics has made possible pharmacogenetic studies of CYP2D6.

CYP2D6 Polymorphisms

Humans shows a wide range of CYP2D6 activities but are conventionally classified as extensive metabolizers (EM) or poor metabolizers (PM) by the ratios of metabolized to unmetabolized drug in urine. PM individuals have a urinary metabolic ration of greater than 12.6 for debrisoquine to 4-hydroxyl-debrisoquine, of greater than 20 for sparteine to 2- and 5-dehyrdrosparteine, and greater than 0.3 for dextromethorphan/dextrophan (see Dayer et al., Enzymatic basis of the debrisoquine/sparteine-type genetic polymorphism of drug oxidation. Characterization of bufuralol 1′-hydroxylation in liver microsomes of in vivo phenotyped carriers of the genetic deficiency, Biochem. Pharmacol. 36, 4145-4152, 1987; and Evans et al., The genetic control of sparteine and debrisoquine metabolism in man with new methods of analysing bimodal distrubtions, J. Med. Genet 20, 321-329, 1983).

The EM phenotype is the normal phenotype that reflects a wildtype genotype. The PM phenotype occurs in about 5-1 0% of the population. The PM phenotype is attributable to a recessive variation in the 2D6 gene. Individuals may be homozygous or heterozygous for CYP2D6 alleles. Heterozygotes carrying a single copy of the wild type allele often exhibit an intermediate level of CYP2D6 activity. Individuals with the PM phenotype have two polymorphisms of the CYP2D6 gene in their diploid genomes, although the polymorphisms need not be the same on each chromosome. Individuals homozygous for null alleles completely lack CYP2D6 activity are and are considered to be phenotypically poor metabolizers (PM). At the other end of the spectrum, UEM (ultra extensive metabolizer) phenotypes have also been identified and can result, from, e.g., duplication of the CYP2D6 gene. For reviews, see Kroemer et al., Life Sci 56:2285-98, 1995; Belpaire et al., Cytochrome P450: genetic polymorphism and drug interactions, Acta Clin Belg 51:254-60, 1996; and Wolf et al., Chapter 18. Cytochrome P450 CYP2D6, IARC Sci Publ 148:209-29, 1999.

The nomenclature for the CYP2D6 polymorphisms has been standardized (see Table 1) (Daly et al., Nomenclature for human CYP2D6 alleles, Pharmacogenetics 6:193-201, 1996). The assay of the invention can be used to assay any of the polymorphisms described herein, and may be applied to any CYP2D6 polymorphism, as well as any other P450 enzyme polymorphism.

The wild type CYPD26 allele is referred to as CYP2D6* 1. Different CYP2D6 phenotypes result from a variety of polymorphisms. These may include, by way of non-limiting example, single nucleotide point mutations (SNPs), including those that occur in the reading frame that encodes CYP2D6 and alter the amino acid sequence of CYP2D6, abberrant RNA splicing, deletions, duplications, inversions, translocations, etc. Some exemplary polymorphisms are as follows.

P450 polymorphisms can result in aberrant RNA splicing that effects P450 expression, usually in a deleterious way. For example, CYP2D6*4 (2D6B) has point mutations in exons 1, 3, 8 and 9, as well as a base change at the third intron splice site that results in aberrant transcript splicing (Gonzales et al., 1988; Kagimoto et al., Multiple Mutations of the Human Cytochrome P450IID6 Gene (CYP2D6) in Poor Metabolizers of Debrisquine, J. Biol. Chem. 265:17209-17214, 1990). Another polymorphism, termed 2D6(F), harbors a mutation that abolishes the splice acceptor site of the first intron and results in a premature stop codon (Marez et al., Polymorphism of the cytochrome P450 CYP2D6 gene in a European population: characterization of 48 mutations and 53 alleles, their frequencies and evolution, Pharmacogenetics 7:193-202, 1997).

P450 polymorphisms can be small or large deletions. CYP2D6*3 (2D6A) has a single nucleotide deletion in exon 5 with a consequent frame shift (Kagimoto et al., 1990). The CYP26*5 (2D6D) is a null allele, i.e., the entire finctional gene is deleted (Gaedigk et al., Deletion of the entire cytochrome P450 CYP2D6 gene as a cause of impaired drug metabolism in poor metabolizers of the debrisoquine/sparteine polymorphism, Am. J. Hum. Genet. 48, 943-950, 1991).

Ultraextensive metabolizer (UTEM) phenotypes can result from the duplication of an active CYP2D6 gene are known (Dahl et al., Ultrarapid hydroxylation of debrisoquine in a Sweddish population. Analysis of the molecular genetic basis, J Pharmacol Exp Ther 274:516-20, 1995; Lovlie et al., Ultrarapid metabolizers of debrisoquine: characterization and PCR-based detection of alleles with duplication of the CYP2D6 gene, FEBS lett. 392:30-34, 1996; Johansson et al., Inherited amplification of an active gene in the cytochrome P450 CYP2D locus as a cause of ultrarapid metabolism of debrisoquine, Proc Natl Acad Sci 90: 11825-11829, 1993).

Daly A. K. et. al., Nomenclature for human CYP2D6 alleles, Pharmacogenetics (1996) 6, 193-201, incorporated by reference herein, provides an exemplary list of CYP2D6 alleles. Preferred VYP2D6 polymorphisms of the present invention are described in Table 1:

TABLE 1
NOMENCLATURE OF CYP2D6 POLYMORPHISMS

| Previous | Present | | Description of | |
|---|---|---|---|---|
| Genotypic | Genotypic | Description of DNA | Amino Acid | CYP2D6 Metabolic |
| Name | Name | Sequence Change | Sequence Change | Phenotype |
| Wildtype | CYP2D6*1 | — | — | Normal |
| | CYP2D6*2 | G1749C, C2938T, and | | Normal |
| | | G4268C | | |
| A | CYP2D6*3 | 2367Δ | Frame-shift | Poor Metabolizer |
| | | | | (PM) |
| B | CYP2D6*4 | G1934A | (splicing affected) | PM |
| D | CYP2D6*5 | (gene deletion) | (null allele) | PM |
| T | CYP2D6*6 | T1795Δ | | PM |
| E | CYP2D6*7 | A3203C | | PM |
| G | CYP2D6*8 | G1846T | | PM |
| | CYP2D6*9 | ΔA2701-A2703 or | | |
| | | ΔG2702-A2704 | | |
| J | CYP2D6*10 | C188T, G1749C, | | PM |
| | | G4268C | | |
| | CYP2D6*11 | G971C | | |
| | CYP2D6*12 | G212A | | |
| | CYP2D6*13 | Hybrid: CYP2D7, | | PM |
| | | exon 1; CYP2D6, | | |
| | | exons 2-9 | | |
| | CYP2D6*14 | G1846A | | PM |
| | CYP2D6*15 | T226 insertion | | PM |
| | CYP2D6*16 | Hybrid: CYP2D7, | | PM |
| | | exons 1-7; CYP2D6, | | |
| | | exons 8-9 | | |
| | CYP2D6*17 | C1111T; G1726C; | | PM |
| | | C2938T; G4268C | | |
| | CYP2D6 | (gene duplication) | (more CYP2D6*¹ | Ultra-extensive |
| | *1x2 | | produced) | metabolizer (UEM) |
| | CYP2D6 | (gene duplication) | (both copies are | UEM |
| | *2x2 | | CYP2D6*2 | |
| | CYP2d6 | (gene duplication) | (both copies are | PM |
| | *4x2 | | CYP2D6*4) | |

Screening for P450 Polymorphisms

The invention is useful for rapidly and simultaneously screening large numbers of samples for several polymorphisms of a cytochrome P450. In an exemplary aspect, a single assay can screen for a CYP2D6 wildtype as well as a variety of CYP2D6 mutations. The mutations include a deletion, a duplication and a base change. Exemplary detectable PM mutations that can be multiplexed include six polymorphisms, one gene deletion (CYP2D6*5) one gene duplication (CYP2D6*4×2). Also included is detection of a gene duplication for the ultraextensive metabolizing phenotype (CYP2D6*1×2 or *2×2).

The alleles detected are preferably one or more of: the wild type allele CYP2D6*1, the deletion allele CYP2D6*5 (2D6D), the gene duplication CYP2D6x2, and the point mutation alleles CYP2D6*3 (2D6A), CYP2D6*4 (2D6B), CYP2D6*6 (2D6T), CYP2D6*7 (2D6E), CYP2D6*8 (2D6G), CYP2D6*10 (D6J), and CYP2D6*17. A specific 4.7 kb PCR product, which cover all the point mutation or wildtype alleles, may also be amplified with CYP2D6 specific primers (Stuven et al., Rapid detection of CYP2D6 null alleles by long distance and multiplex polymerase chain reaction. (1996) Pharmacogenetics 6:417-421.). Another specific 5 kb PCR product, which also covers all the point mutation or wildtype alleles may also be amplified with CYP2D6 specific primers. (Kashuba ADM, et al. Quantification of intraindividual variability and the influence of menstrual cycle phase on CYP2D6 activity as measured by dextromethorphan phenotyping. Pharmacogenetics. (1998) 8:403-410.) These two PCR products can ensure the minimal effect to the primers by the template polymorphism.

A specific 3.5 kb PCR product is amplified with deletion-specific primers when the deletion allele is present, whereas a specific 3.2 kb PCR product is amplified with specific intergenic primers for the duplicated region when the gene duplication is present.

The above PCR products are combined, and the mixture is treated, e.g., with Shrimp Alkaline Phosphatase (SAP) and Exonuclease I, to remove excess dNTPs and PCR primers. This is followed by the single nucleotide primer extension SNaPshot reaction (Lindblad-Toh et al., Large-scale discovery and genotyping of single-nucleotide polymorphisms in the mouse. Nature Genet 2000 Apr. 24(4):381-6). In this reaction, an oligonucleotide primer is designed to have a 3′ end that is one nucleotide 5′ to a specific point mutation site or to a specific sequence found at a boundary of a mutation that occurs at a larger scale (e.g., a duplication, deletion, inversion, etc.). The primer hybridizes to the PCR amplicon in the presence of fluorescently labeled ddNTPs and a DNA polymerase. The polymerase extends the primer by one nucleotide, adding a single, labeled ddNTP to its 3′ end. Each dideoxynucleotide (e.g., ddATP, ddGTP, ddCTP, ddTTP, ddUTP, etc.) is differently labelled, e.g., each is labeled with a different fluorescent colored dye. The primers are tagged with varying lengths of nonspecific polynucleotides (e.g., poly-GACT) to allow multiplex detection of 5 or more, preferably 10 or more, and most preferably 100 or more different mutations (polymorphisms) in a single reaction. Excess ddNTPs are removed from the reaction mixture by SAP treatment. The products are fluorescently labeled oligonucleotides, each one of which is detected, for example using an automated DNA sequencer (e.g., ABI PRISM 3100 Genetic Analyzer) based on both its size (determined by electrophoretic mobility) and its respective fluorescent label.

The invention provides compositions and methods for high-throughput assay for detecting mutations in cytochrome P450 genes, particularly the human CYP2D6 gene. The assay can detect multiple polymorphisms using only two PCR reactions. In the Examples, six point mutations in the CYP2D6 gene, a complete deletion of the CYP2D6 gene, and a gene CYP2D6 duplication, are assayed according to the assays of the invention. The assay of the invention detects a variety of polymorphisms at a specific nucleotide position, or at multiple nucleotide positions, in a single run.

### EXAMPLES

The following examples serve to illustrate the present invention. These examples are in no way intended to limit the scope of the invention.

### Example 1

Preparation of Biological Samples

Biological samples and other specimens are obtained, stored and prepared for assay using protocols that may vary depending on the type of sample that is to be used in the assay. Representative protocols for the preparation of different types of specimens include the following non-limiting examples.

1.1 Obtaining Specimens from Different Sources

1.1.1 Whole Blood: Collect 5cc of whole blood in a lavender-top (EDTA) tube or yellow-top (ACD) tube. Green-top (Na Heparin) tubes are acceptable but not recommended.

1.1.2 Cultured cells: Send two T25 culture flasks with about 80% to about 100% confluent growth.

1.1.3 Tissue Samples: Collect a 1 cm×1 cm tissue sample in a sterile container. Do not use fixative.

1.1.4 Pediatric Sample: Collect 2 cc of whole blood in a lavender-top (EDTA) tube.

1.1.5 Bone Marrow: Collect samples using protocols described in Oliver et al. (Use of Single Nucleotide Polymorphisms (SNP) and Real-Time Polymerase Chain Reaction for Bone Marrow Engraftment Analysis, J Mol Diagn 2:202-208, 2000).

1.2 Transport, Storage, and Stability

1.2.1 Whole blood is shipped at room temperature (15°-30° C.), stored at 2-8° C. and should not be frozen. Samples of whole blood are stable for 4 weeks at 2-8° C.

1.2.2 Cultured cells are shipped at room temperature (151-30° C.), and should not be refrigerated or frozen. Cultured cells are stored at room temperature (15°-30° C.) and are stable for 24 hours.

1.2.3 Tissue samples are stored at −60 to −80° C. and are stable for one year.

1.2.4 Amniotic cells are stored at 2-8° C. after an aliquot is removed for culturing. Amniotic fluid and chorionic villi should not be refrigerated or frozen.

1.2.5 DNA prepared from samples within 24 hours of receipt is stable for 5 years at 2 to 8° C.

### Example 2

Instruments and Equipment

The following commercially available instruments and equipment are non-limiting examples of those that may be used to practice the invention. Those skilled in the art will be able to determine other instruments and equipment that may be used in the methods of the invention.

2.1 Pipettes

Standard pipettes are used to deliver volumes ranging from 0.5 to 100 ml. For volumes less than 1 ml, pipettors such as the P-10, P-20, P-200, P-1000 (Rainin Instruments, LLC) pipettors are used. Pipet tips are selected from Barrier Pipet Tips (Robbins Scientific); Pipet Tips, 20 μl and 250 μl (Beckman), and ART (aerosol resistant tips) for P-10, P-20, P-200, P-1000) (Rainin).

2.2 Electrophoresis

Examples of apparatuses that may be useful for electrophoresis and visualization are an agarose gel electrophoresis apparatus, such as CBS Scientific horizontal mini-gel; a power supply having a constant voltage of 200V or better variable power supply for electrophoresis, such as the BioRad Model 200; photodocumentation apparatus, such as the Alpha Innotech Alphalmager or Polaroid DS34 t; and a transilluminator, e.g., a VWR Model LM-20E or equivalent.

2.3 Centrifugation

Centrifugation is carried in BioMek 2000 or Vortex (VWR; G-560) instruments and centrifuges for spinning PCR trays (Sorvall T6000D). ). The 96-well-plate centrifugation system from Qiagen may also be used. Microcentrifuges such as those from Eppendorf are used with Microcentrifuge tubes (from, e.g., National Scientific, CN065S-GT).

2.4 PCR Containers and Reaction Plates

For DNA amplification (PCR), 2 ml MicroTubes with screw caps (Sarstedt; 72.693-005) may be used. A variety of 96-well plates suitable for PCR and other manipulations can be used. In the Examples herein, ABI MicroAmp Optical 96-well Reaction Plates (P/N#N801-0560) are used with ABI 96-well Plate Septa (PIN#4315933), or Microseal 96-well PCR microplates (MJ Research, MSP-9601) are used with Microseal A sealing film for microplates (MJ Research, MSA-5001). A 96-place storage system exemplified by VWR #30128-330, is used to store plates containing samples between steps in the assay.

2.5 PCR Cycler

A PCR cycler capable of processing 96-well plates is used in the Examples. Exemplary PCT thermal cyclers include the GeneAmp 9600 (Perkin-Elmer) or the PTC 200 (MJ Research). The MJR PTC 200 has features that are desirable regardless of which instrument is used: heating rates of up to 3° C./second, which reduce reaction times, and rapid temperature homogeneity (e.g., ±0.4° C. within 30 seconds at 90° C.). The heating block that is used may be, for example, VWR's Heat Block (VWR, 13259-007).

2.6 Automated Laboratory Workstation

In order to process a large number of samples for CYP2D6 genotyping, a multipurpose automated or semi-automated programmable workstation is used (Meldrum, Automation for Genomics, Part One: Preparation for Sequencing, Genome Research, 10:1081-1092, 2000; Meldrum, Automation for Genomics, Part Two: Squencers, Microarrays, and Future Trends, Genome Research, 10:1288-1303, 2000). Preferred features of the workstation include the ability to rapidly and accurately pipette, dilute and dispense small volumes of liquids. The exemplary programable workstation used herein is the BioMek® 2000 (Beckman Coulter, Inc.).

2.7 Capillary Electrophoresis DNA Sequencer

For high throughput of PCR products, an automated capillary electrophoresis (CE) system is used in order to separate labeled DNA molecules in a size-dependent manner, so that signals corresponding to each nucleotide in a sequence are detected in a sequential fashion. For reviews of the use of CE in DNA sequencing and polymorphism analysis, see Heller, Electrophoresis 22:629-43, 2001; Dovichi et al., Methods Mol Biol 167:225-39, 2001; Mitchelson, Methods Mol Biol 162:3-26, 2001; and Dolnik, J Biochem Biophys Methods 41:103-19, 1999. In the Examples, the ABI PRISM® 3100 Genetic Analyzer is used with an ABI PRISM 3100 capillary array, 36-cm (P/N#4315931). This provides a multi-color fluorescence-based DNA analysis system that uses capillary electrophoresis with 16 capillaries operating in parallel to separate labeled PCR products. A CE DNA sequencer/analyzer that operates 96 capillaries may be preferable in assays wherein 96-well plates are used. Analyzers with the capacity to process 96 wells include the MegaBACE™ 1000 DNA Analysis System (Molecular Dynamics, Inc and Amersham Pharmacia Biotech) and the 3700 DNA Analyzer from (Perkin-Elmer Biosystems)

### Example 3

Reagents

3.1 Stock Reagents

The following exemplary stock reagents are used and are stable for the indicated times when stored at the indicated temperature/conditions.

3.1.1 Agarose, SeaKem GTG (FMC 50074). Store ambient (18° C.-26° C.), stable for 1 year.

3.1.2 dNTP set, ultrapure, 100 mM solution (Pharmacia 27-2035-01). Store at −10° C. to -30° C., stable for 1 year.

3.1.3 EDTA, disodium (Sigma E-5134). Store ambient (18° C.-26° C.), stable for 1 year.

3.1.4 Ethidium bromide (Life Technologies 15585-011). Store ambient (18° C.-26° C.), stable for 1 year.

3.1.4 Ficoll (Sigma, Cat. #F2637). Store at 18-25° C. stable for 1 year.

3.1.5 Bromophenol Blue (Sigma, cat. #B6131). Store at 18-25° C. stable for 1 year.

3.1.6 Xylene Cyanol (Kodak, cat. #IB72120). Store at 18-25° C. stable for 1 year.

3.1.7 0.5 MEDTA, pH 8.0 (Amresco, cat. #E177), Store at 18-25° C. stable for 1 year.

3.1.8 Taq Extender PCR additive (Stratagene 600148) stored at −20° C. stable for 1 year.

3.1.9 If a commercially available DNA extraction kit is not used, reagents for the Proteinase K or phenol-chloroform extraction method should be prepared as is known in the art.

3. 1.10 ABI 3100 POP-4 polymer (P/N4316335), stable for 1 year when stored at 2 to 10° C.

3.2 Stock Solutions

The following exemplary stock solutions are used and are stable for the indicated times when stored at the indicated temperature/conditions.

3.2.1 Water, molecular biology grade (BioWhittaker 16-001Y or equivalent) stored ambient (18° C.-26° C.), stable for 1 year.

3.2.2 6×Gel loading dye (no xylene cyanol)

3.2.3 100 mM disodium EDTA pH 8.0

3.2.4 6%-12% (w/v) Ficoll 400

3.2.5 0.25% (w/v) bromophenol blue

3.2.6 10× TBE buffer

3.2.6.1 Prepare as 890 mM Tris Base, 890 mM Boric Acid, and 20 mM Disodium EDTA

3.2.6.2 TBE buffer (Amresco 0658 or equivalent) stored ambient (18° C.-26° C.), stable for 1 year.

3.2.7 ABI 10× Buffer (P/N402824), stored at 2 to 10° C., stable for 1 year.

3.2.8 ABI Hi Di Formamide (P/N4311320). Stored at −10° C. or colder, stable for 1 year or until the indicated expiration date.

3.2.10 100× TE buffer (Sigma T-9285 or equivalent) stored ambient (18° C.-26° C.), stable for 1 year.

3.2.11 ABI 5× Sequencing Buffer, PE Applied Biosytems, (P/N4305603), stored at −15° C. to −25° C. stable for 1 year.

3.3 Kits

The following exemplary kits may be used and are stable for the indicated time when stored at the indicated temperature/conditions.

3.3.1 ABI SNAPshot multiplex kit (P/N4323161), stored at −10 to −30° C. stable for 6 months

3.3.2 HotStarTaq™ PCR Core Kit (Qiagen 203203 or 203205) (HotStarTaq™ enzyme, 25mMg⁺⁺, M10X buffer & 5× Q Solution), stable for 1 year when stored at −10° C. to −30° C.

3.4 Enzymes

The following exemplary enzymes may be used and are stable for the indicated time when stored at the indicated temperature/conditions.

3.4.1 Shrimp Alkaline Phosphatase (USB Corporation, P/N70092X), stable for 6 months when stored at −10 to −30° C.

3.4.2 Exonuclease I (USB Corporation, P/N70073X), stable for 6 months when stored at −10 to −30° C.

3.5 Standards

The following exemplary standards may be used and are stable for the indicated time when stored at the indicated temperature/conditions.

3.5.1 DNA ladder, 500 bp (Gibco BRL 10594-018) stable for 1 year when stored at −20° C.

3.5.2 ABI GeneScan-120 LIZ Size Standard (P/N4322362), stable for six months when stored at 2 to 10° C.

3.6 PCR Amplification Primers

Oligonucleotides used as PCR primers were prepared by Operon Technologies, Inc. (0.05, 0.2 or 1.0 micromole scale synthesis, no HPLC purification) and stored as 100 μM stocks at −10° C. or colder, conditions under which they are stable for 1 year. Table 2 gives the sequences of PCR primers used in the Examples.

TABLE 2

PCR PRIMER SEQUENCES

| SEQ | Primer | Primer | |
|---|---|---|---|
| ID NO: | Location/Description | Name | Primer Sequence |
| 1 | 2D6 gene | 2D6F1 | 5′- GGTAAGGGCCTGGAGCAGGAA -3′ |
| 2 | 2D6 gene | 2D6R2 | 5′- GCCTCAACGTACCCCTGTCTC -3′ |
| 3 | Gene Deletion | 2D6DF | 5′- ACCGGGCACCTGTACTCCTCA -3′ |
| 4 | Gene Deletion | 2D6DR | 5′- GCATGAGCTAAGGCACCCAGAC -3′ |
| 5 | Gene Duplication | CYP207F | 5′- CCCTCAGCCTCGTCACCTCAC -3′ |
| 6 | Gene Duplication | CYP32R | 5′- CACGTGCAGGGCACCTAGAT -3′ |
| 7 | 2D6 gene | 2D6F3 | 5′- CCAGAAGGCTTTGCAGGCTTCA -3′ |
| 8 | 2D6 gene | 2D6R4 | 5′- ACTGAGCCCTGGGAGGTAGGTA -3′ |

3.7 Primer Extension Primers

Primer extension primers were prepared by Operon Technologies, Inc. (0.05, 0.2 or 1.0 micromole scale synthesis, HPLC purification): stored as 100 μM stocks at −10° C. or colder, stable for 1 year. Table 3 shows the sequences of primers used in the Examples.

TABLE 3

PRIMER EXTENSION SEQUENCES

| | | | |
|---|---|---|---|
| | | Primer | |
| SEQ ID NO: | Mutation | name | Primer Sequence |

| | | | |
|---|---|---|---|
| 9 | CYP2D6*4 | SNP11 | 5′-CGCATCTCCCACCCCCA-3′ |
| 10 | CYP2D6*8 | SNP12 | 5′-GACTGCCTTCGCCAACCACTCC-3′ |
| 11 | CYP2D6*Nx2 | SNP13 | 5′-GACTCAGCCTCGTCACCTCACCACAG-3′ |
| 12 | CYP2D6*6 | SNP14Δ | 5′-ACTGACTGACTCGGCCTCCTCGGTCACCC-3′ |
| 13 | CYP2D6*7 | SNP15 | 5′-CTGACTGACTGGGCCTCCTGCTCATGATC |
| | | | CTAC-3′ |
| 14 | CYP2D6*5 | SNP16 | 5′-CTGACTGACTGACTCTCTTGTTGACCAGG |
| | | | CTGGAGTG-3′ |
| 15 | CYP2D6*3 | SNP17 | 5′-TGACTGACTGACTGACTCTGGATGAGCTG |
| | | | CTAACTGAGCAC-3′ |
| 16 | CYP2D6*10 | SNP19Δ | 5′-CTGACTGACTGACTGACTGACTCGCCAAC |
| | | | GCTGGGCTGCACGCTAC-3′ |
| 17 | CYP2D6*17 | SNP30Δ5 | 5′-TGACTGCCCGAAACTCAGGATCTGG-3′ |
| 18 | CYP2D6*17 | SNP31 | 5′-CTGACTGACTGACTAGAACAGGTCAGCCA |
| | | | CCACTATGC-3′ |
| 19 | CYP2D6*17 | SNP30Δ6 | 5′-TGACTGCCCAAAACTCAGGATCTGG-3′ |

3.8 Working Stocks for PCR, Primer Extension, and SAP Treatment

3.8.1 CYP2D6 and CYP2D6D Duplex PCR

3.8.1.1 5× Primer Mix for CYP2D6 and CYP2D6D Duplex PCR is prepared according to the following recipe and is stable for 1 year when stored at −70° C.

| Primer (100 μM) | Volume | [5X] | [working] |
|---|---|---|---|
| 2D6F1 | 19.5 μl | 1.50 μM | 0.300 μM |
| 2D6R2 | 19.5 μl | 1.50 μM | 0.300 μM |
| 2D6DF | 19.5 μl | 1.50 μM | 0.300 μM |
| 2D6DR | 19.5 μl | 1.50 μM | 0.300 μM |
| H2O: | 1222.0 μl | | |
| Total: | 1300.0 μl | | |

3.8.1.2 Long PCR CYP2D6 and CYP2D6D Duplex Mix is prepared according to the following recipe.

| Components | For 114 Rxns |
|---|---|
| 10X Qiagen PCR Buffer | 285.0 μL |
| 5X Q Solution | 570.0 μL |
| 25 mM dNTP mix | 28.5 μL |
| 5X primer mix (2D6&2D6D) [3.8.1.1, above] | 570.0 μL |
| H2O | 1100.1 μL |
| Total | 2553.6 μL |

Aliquots of 492.8 μL (enough for 22 reactions) are each placed in a 1.7 ml tube. The aliquots are stable for 3 months when stored at −70° C. To account for pipetting variability, one tube is used for each setup of 20 reactions.

3.8.2 CYP2D6 and CYP2D6×2 PCR

3.8.2.1 5× Primer Mix for CYP2D6 and CYP2D6×2 PCR is prepared according to the following recipe and is stable for 1 year when stored at −70° C.

| Primer (100 μM) | Volume | [5X] | [working] |
|---|---|---|---|
| 2D6F3 | 19.5 μl | 1.50 μM | 0.300 μM |
| 2D6R4 | 19.5 μl | 1.50 μM | 0.300 μM |
| 207F | 26.0 μl | 2.00 μM | 0.400 μM |
| 32R | 26.0 μl | 2.00 μM | 0.400 μM |
| H2O: | 1209.0 μl | | |
| Total: | 1300.0 μl | | |

3.8.2.2 Long PCR: CYP2D6 and CYP2D6×2 PCR Mix is prepared according to the following recipe.

| Components | for 114 Rxns |
|---|---|
| 10X Qiagen PCR Buffer | 285.0 μL |
| 5X Q Solution | 570.0 μL |
| 25 mM dNTP mix | 28.5 μL |
| 5X primer mix (2D6 and 2D6 × 2) | 570.0 μL |
| [3.8.2.1, above] | |
| H2O | 1100.1 μL |
| Total | 2553.6 μL |

Aliquots of 492.8 SL (enough for 22 reactions) are each placed in a 1.7 ml tube. The aliquots are stable for 3 months when stored at −70° C. To account for pipetting variability, one tube is used for each setup of 20 reactions.

3.8.3 25 mM dNTPs Stock Solution

The 100 mM stock solutions of dATP, dCTP, dGTP, dTTP are thawed, and 50 μl of each is added to a sterile microfuge tube. The tube is vortexed for 2 sec to mix, and then spun in a microcentrifuge at maximum speed for 2 sec. The 25 mM dNTPs Stock Solution is stored at −20° C. or −80° C. and should not be thawed and refrozen more than three times.

3.8.4 SAP+ExoI Cocktail

Combine 5 μl of SAP (1 unity) and 0.2 μl of ExoI (10 unit/μl) in 1× SAP buffer to a final volume of 15 μl per reaction. The SAP+ExoI Cocktail is prepared fresh before each use.

| | Concentration | Volume (μl) for 120 rxns (full plate) |
|---|---|---|
| SAP | 1 unit/μl | 600 |
| Exo I | 10 unit/μl | 24 |
| 10x SAP buffer | 10x | 240 |
| Sterile H20 | | 936 |
| Total | | 1800 |

3.8.5 Primer Extension Primer Mix is prepared according to the following recipe.

| | | | | Volume |
|---|---|---|---|---|
| | | | | of |
| | | | Volume of | primer |
| | | | primer added | added |
| | Concentration | Volume of primer | (115 rxns-full | (large |
| Primer | (μM) | added (1 rxn) | plate) | scale) |
| SNP11 | 100 | 0.02 μl | 2.3 μl | 40 μl |
| SNP12 | 100 | 0.02 μl | 2.3 μl | 40 μl |
| SNP13 | 100 | 0.02 μl | 2.3 μl | 40 μl |
| SNP14Δ | 100 | 0.04 μl | 4.6 μl | 80 μl |
| SNP15 | 100 | 0.02 μl | 2.3 μl | 40 μl |
| SNP16 | 100 | 0.08 μl | 9.2 μl | 160 μl |
| SNP17 | 100 | 0.02 μl | 2.3 μl | 40 μl |
| SNP19Δ | 100 | 0.08 μl | 9.2 μl | 160 μl |
| SNP30Δ5 | 100 | 0.02 μl | 2.3 μl | 40 μl |
| SNP31 | 100 | 0.02 μl | 2.3 μl | 40 μl |
| dH2O | | 0.66 μl | 75.9 μl | 1320 μl |
| Total: | | 1.0 μl | 115 μl | 2000 μl |

The mix is prepared in 15 ml sterile conical tubes and dispensed in 1 to 1.5 ml aliquots per microcentrifuge tube and stored at −70° C. or colder.

3.8.6 SNaPshot Primer Extension Master Mix

Five (5) μl of ABI SNaPshot Ready Mix, 1 μl of Primer Extension Primer Mix and 1 μl Sterile H20 are combined to a final volume of 7 μl per reaction. The Mix is prepared fresh before each use, and kept on ice until used.

| Reagent | Per Well | Per Plate* |
|---|---|---|
| SnaPshot Ready Mix | 5 μl | 560 μl |
| Extension Primer Mix | 1 μl | 112 μl |
| DH₂O | 1 μl | 112 μl |
| Total | 7 μl | 784 μl |

*Contains extra for aliquot by BioMek 2000.

3.8.7 SAP Cocktail:

For each reaction, 1 μl of SAP (1 unit/μl) and 1 μl of water are combined to a final volume of 2 μl. The SAP cocktail is freshly prepared before each use.

| Reagent | Per Well | Per Plate* |
|---|---|---|
| SAP | 1 μl | 140 μl |
| Dh2O | 1 μl | 140 μl |
| Total | 2 μl | 280 μl |

*Contains extra for aliquot by BioMek 2000.

3.8.8 Loading Mix: Ten (10) μl of Hi-Di Formamide and 0.5 μl GeneScan 120 LIZ Size Standard are combined to a final volume of 10.5 μl per sample. Lodging Mix is prepared fresh before each use.

| Reagent | Per Well | Per Plate* |
|---|---|---|
| Hi-Di Formamide | 10 μl | 1120 μl |
| GeneScan 120 LIZ Size Standard | 0.5 μl | 56 μl |
| Total | 10.5 μl | 1176 μl |

*This setup is for a full 96 well plate.

### Example 4

Procedure

4.1 Preparation of Sample Trays

4.1.1 CYP2D6 and CYP2D6D PCR Sample Tray

PCR master mix (CYP2D6 and CYP2D6D Duplex Mix) is prepared according to Example 3.8.1.2 and is used in the reaction. The following table describes a recipe that results in a sufficient volume for a full PCR plate (sample tray; 96-wells), and allows for excessive solution to enable pipetting from a trough with an 8-channel pipettor into all PCR wells.

| | | | Cocktail × 56 | | Cocktail × 112 | |
|---|---|---|---|---|---|---|
| | 1 Rxn | | (1/2 plate) | | (full plate) | |
| Master Mix [3.8.1.2] | 22.4 | μL | 1254.4 | μL | 2508.8 | μL |
| HotStarTaq | 0.3 | μL | 16.8 | μL | 33.6 | μL |
| Taq Extender | 0.3 | μL | 16.8 | μL | 33.6 | μL |

| | | | | |
|---|---|---|---|---|
| Qiagen DNA* | 2.0 | μL | — | — |
| Total | 25 | μL | | |

*This recipe is for PCR setup in a 96 well plate format. If a DNA sample is extracted with the phenol/chloroform method, it should be diluted in sterile water to a concentration of 20-40 μg/ml.

5.1.1.2 CYP2D6 and CYP2D6×2 PCR Sample Tray

PCR master mix (CYP2D6×2 PCR Mix) is prepared according to Example 3.8.2.2 and is used in the reaction. The following table describes a recipe that results in a sufficient volume for a full PCR plate (sample tray), and allows for excessive solution to enable pipetting from a trough with an 8-channel pipettor into all PCR wells.

| | Cocktail × 56 | | | | Cocktail × 112 | |
|---|---|---|---|---|---|---|
| 1 Rxn | (1/2 plate) | | | | (full plate) | |
| Master Mix [3.8.2.2] | 22.4 | μL | 1254.4 | μL | 2508.8 | μL units |
| HotStarTaq | 0.3 | μL | 16.8 | μL | 33.6 | μL |
| Taq Extender | 0.3 | μL | 16.8 | μL | 33.6 | μL |

| | | | | |
|---|---|---|---|---|
| Qiagen DNA* | 2.0 | μL | — | — |
| Total | 25 | μL | | |

*This recipe is for PCR setup in a 96 well plate format. If a DNA sample is extracted with the phenol/chloroform method, it should be diluted in sterile water to a concentration of 20-40 μg/ml.

4.2 PCR Reactions

For automated PCR setup on the BioMek 2000 robotic workstation, the PCR tray, a box of Robbins 125 μL pipet tips, a box of 20 μL pipet tips, the Qiagen sample tray and the reagent reservoir (trough) are placed at the appropriate positions on the BioMek work surface. If the PCR or subsequent steps are set up manually, the same master mix recipe/digestion recipe is used, and the assay proceeds as described below without the BioMek, and single or multichannel pipettors and tips are used.

The master mix is added to the reagent reservoir. Eight positions at the end of the Qiagen sample tray are left open for controls. The sample tray is briefly spun down in a plate centrifuge outside of the master mix and template addition area (i.e., in a clean room). The control samples (typically, four positive and two negative controls) are placed in the appropriate positions in the sample tray.

The BioMek station first pipets 23 μl of the master mix into each 0.2 ml PCR tray wells, and then adds 2 μl specimen DNA or control. The wells are tightly sealed with PCR tube caps or Microseal A film. The sample tray is briefly (˜5 s) vortexed and spun down for about 30 s in a plate centrifuge at 2,000-6,000g (1,600 rpm in a Sorvall T6000D centrifuge).

The cycling program (below) is started on a thermal cycler such as the MJR PTC 200. When the temperature reaches >85° C. the PCR tray is placed in the thermal cycler and its lid is sealed.

The cycling parameters for 2D6 and 2D6D PCR are:

| Step | Temperature | Time |
|---|---|---|
| 1 | 95° C. | 15 min. |
| 2 | 94° C. | 10 sec |
| 3 | | 0.60/sec. Ramp |
| 4 | 55° C. | 15 sec. |
| 5 | | 0.60/sec. Ramp |
| 6 | 72° C. | 15 sec. |
| 7 | | 0.80/sec. Ramp |
| 8 | [Go to step 2 and repeat for 31 cycles*] | |
| 9 | 72° C. | 5 min. |
| 10 | 4° C. | Hold |

*Typically, 31 cycles is optimal, however 29-33 cycles may be used if the PCR products from 31 cycles are less than optimal.

The cycling parameters for 2D6 and 2D6×2 PCR are:

| Step | Temperature | Time |
|---|---|---|
| 1 | 95° C. | 15 min. |
| 2 | 94° C. | 10 sec |
| 3 | 65° C. | 1 min. |
| 4 | 68° C. | 5 min. |
| 5 | Goto step 2: | 9 more times |
| 6 | 94° C. | 10 sec. |
| 7 | 65° C. | 1 min. |
| 8 | 68° C. | 5 min + 10 sec./cycle. |
| 9 | Goto step 6: | 29 more times* |
| 10 | 72° C. | 5 min |
| 11 | 4° C. | Hold |
| 12 | | End |

*Typically, 31 cycles is optimal, however 29-33 cycles may be used if the PCR products from 31 cycles are less than optimal.

After PCR is complete, the products may be stored refrigerated up to one week or frozen (<−10° C.) if a longer storage period is necessary, or they may be used immediately in the following procedures.

4.3 First SAP and Exol Digestion

Digestion starts by adding 3 μl of CYP2D6 and CYP2D6D Duplex Mix PCR product and 2 μl of CYP2D6 and CYP2D6×2 PCR Mix PCR product to 15 μl of the SAP+ExoI Cocktail. The plate is sealed, vortexed and spun down in the plate centrifuge. The plate is then placed in the MJR PTC 200 thermal cycler and a cycling program is run using the following parameters.

| Step | Temperature | Time |
|---|---|---|
| 1 | 37° C. | 2 hr. |
| 2 | 75° C. | 15 min. |
| 3 | 4° C. | Hold |

Each step uses rapid (default) ramp to reach desired temperature. The SAP/Exol—treated PCR products can be stored at 2-8° C. until use.

4.4 Primer Extension

SNaPshot Primer extension Master Mix is freshly prepared according to Example 3.8.6, and 3 μl of the master mix is added to 3 μl of digestion product from Example 4.3. After addition of the SnaPshot Primer Extension Master Mix, each plate is immediately placed in the thermocycler an the “SNAPSHOT” program is immediately run.

The plate should not be allowed to sit at room temperature more than 30 seconds. The plate is sealed, vortexed and spun down in the plate centrifuge. The plate is then placed in the MJR PTC 200 thermal cycler and a cycling program is run using the following parameters.

| Step | Temperature | Time |
|---|---|---|
| 1 | 96° C. | 10 sec. |
| 2 | 50° C. | 5 sec. |
| 3 | 60° C. | 30 sec. |
| 4 | | Go to step 2 24 more times. |
| 5 | 4° C. | Hold |

Each step uses rapid (default) ramp to reach desired temperature. The reaction plates are stored at 2-8° C. until use.

4.5 Second SAP Digestion

2 μl of th SAP Cocktail is mixed with 10 μl primer extension product from Example 4.4. The plate is sealed and vortexed, and then spun down in the plate centrifuge. The plate is placed in the MJR PTC 200 thermal cycler and a cycling program is run using the following parameters.

| Step | Temperature | Time |
|---|---|---|
| 1 | 37° C. | 1 hr. |
| 2 | 75° C. | 15 min. |
| 3 | 4° C. | Hold |

Each step uses rapid (default) ramp to reach desired temperature. The digestion plate is stored at −15° C. or lower until use.

4.6 Electrophoresis on ABI 3100 Genetic Analyzer

SAP-digested samples are prepared according to Example 4.5 for loading using a BioMek 2000. The SNaPShot product is diluted 15-fold with water, and then 2 μl of the diluted product is mixed with 10.5 μl of the Loading Mix. The plate is covered with septa, vortexed and spun down in the plate centrifuge. The plate is heated at 95° C. for 5 minutes, then immediately placed on ice for 3 minutes or until use. The plate is spun down in a plate centrifuge to collect condensation. The plate is then assembled and loaded onto the ABI3100 Genetic Analyzer.

### Example 5

Other Cytochrome P450 Enzymes

In the preceding Examples, assays for polymorphisms of CYP2D6 are described. The invention may be applied to any set of polymorphisms of other cytochrome P450 enzymes. These include, but are not limited to, CYP1A1, CYP1A2, CYP2A6, CYP2B6, CYP2C9, CYP2C18, CYP2C19, CYP2D6, CYP2E1, CYP3A4, and CYP3A5. Cytochrome P450 enzymes of particular interest include the following.

5.1 CYP1A1

CYP1A1 is also known as aryl hydrocarbon hydroxylase, catalyses the first step in the metabolism of polycyclic aromatic hydrocarbons to carcinogens. CYP1A1 is an inducible enzyme that is important for conversion of carcinogenic polycyclic aromatic hydrocarbons to epoxides. A phenotype polymorphism in inducibility was first described in 10% of Caucasians who showed much higher CYP1A1 activity in lymphocytes after exposure to inducer than was observed in the rest of the study group (Kellerman et al., Arylhydrocarbon hydroxylase and bronchogenic carcinoma, New Eng. J. Med. 289, 934-937, 1997).

In addition to the CYP1A1*1 (wildtype) allele, several polymorphisms are known, including by way of non-limiting example. CYP1A1*2A (T3801C), CYP1A1*2C (G4889A, which results in the amino acid substitution 1462V, which is positioned in the heme binding region), CYP1A1*3 (T3205C), CYP1A1*4 (C4887A, which results in the amino acid substitution T461N), and T6235C, which lies outside the coding region in the 3′-flanking region. The T6235C polymorphism may be associated with increased inducibility.

5.2 CYP1A2

CYP1A2 metabolizes tricyclic antidepressants (TCAs), Propranolol, F-Warfarin, and Theophylline. CYP1A2 is known to be affected by tobacco smoking. For example, smoking induces formation of CYP1A2 enzymes causing smokers to require higher doses of theophylline than non-smokers. In addition to the CYP1A2*1 (wildtype) allele, several polymorphisms are known, including by way of non-limiting example CYP1A2*1C (G3858A) and CYP1A2*1F (C164A).

5.3 CYP2A6

CYP2A6 is known to catalyse the 7-hydroxylation of coumarin and nicotine. Some evidence of bimodality of its metabolism in vivo has been obtained from phenotyping studies (Cholerton et al., Comparison of a novel thin-layer chromatogrphic-fluorescence detection method with a spectrofluorometric method for the determination of 7-hydroxycoumarin in human urine, J. Chromatogr. 575, 325-330, 1992; Rautio et al., Interindividual variation of coumarin-hydroxylase in healthy volunteers, Pharmacogenetics 2, 227-233, 1992). The organization and structure of the CYP2A6 gene cluster has been characterized (Hoffman et al., Organisation and evolution of the cytochrome P450 2A6-2B-2F subjfamily gene cluster on human chromosome, J. Molec. Evolut. 41, 894-900, 1995), and at least three alleles of the CYP2A6 gene were found, i.e., wild-type (CYP2A6*1) and two polymorphisms, CYP2A6*2 (CYP2A6vl) and CYP2A6*3 (CYP2A6v2) (Fernandez-Salguero et al., A genetic polymorphiism in coumarin 7-hydroxylation: sequence of the human CYP2A6 genes and identification of variant CYP2A6 alleles, Am. J. Hum. Genet. 57, 651-660, 1995). CYP2A6*2 has a point mutation causing the amino acid change L160H, and CYP2A*3 has several alterations in exons 3, 6 and 8 generated apparently by gene conversion between CYP2A6 and CYP2A7. A deletion of CYP2A6 is also known (Nunoya et al., A new deleted allele in the human cytochrome P450 2A6 (CYP2A6) gene found in individuals showing poor metabolic capacity to coumarin and (+)-cis3,5-dimethyl-2(3-pyridyl)thiazolidin-4-one hydrochloride (SM-12502), Pharmacogenetics 8, 239-249, 1998). All of these polymorphisms are considered to be inactive enzymatically.

5.4 CYP2C9

CYP2C9 is important in the metabolism of ibuprofen, tetrahydrocannabinol, S-warfarin, tolbutamide mefenamic acid, losar tan, diclofenac7, phenytoin and NSAIDS (Goldstein and deMorais, Biochemistry and molecular biology of the human CYP2C subfamily, Pharmacogenetics 4, 285-299, and Identification of new genetic defect responsible for the polymorphism of S-mephenytoin metabolism in, Japanese Mol. Pharmacol. 46, 594-598, 1994).

In addition to the CYP2C9* 1 (wildtype) allele, several polymorphisms are known, including by way of non-limiting example, CYP2C9*2 (RI44C) and CYP2C9*3 (1359L) (Stubbins et al., Genetic analysis of the human cytochrome P450 CYP2C9 locus, Pharmacogenetics 6:429-439, 1996). The *2 and *3 variants produce intact enzyme with reduced enzymatic activity arising from amino acid substitutions that are at positions critical for activity. Allele frequencies for these two variants are of the order of 6-12% for Caucasian populations (Sullivan-Klose et al., The role of the CYP2C9-Leu 359 allelic variant in the tolbutamide polymoprhism, Pharmacogenetics 6:341-349, 1996; Stubbins et al., Genetic analysis of the human cytochrome P450 CYP2C9 locus, Pharmacogenetics 6, 429-439, 1996; Yasar et al., Genetic analysis of CYP2C9 polymorphism in a Swedish population, In: Proceedings of the 12th International Symposium on Microsomes and Drug Oxidations, Montpelier, France, 20-24, Jul. 1998; Ackerman et al., A novel CYP2C9 intron 2T/C transition and linkage of mutations Cysl44 and Leu 359, Proceedings of the 12th International Symposium on Microsomes and Drug Oxidations, Montpelier, France, 20-24, 1998). In studies of Chinese and Japanese populations, CYP2C9*2 was not detected while CYP2C9*3 occurred at frequencies around 2% (Wang et al., Detection of CYP2C9 polymoprhiism based on the polymerase chain reaction in Chinese, Pharmacogenetics, 5,37-42, 1995; Nasu et al., Genetic analysis of CYP2C( polymoprhism in a Japanese population, Pharmacogenetics 7, 405-409, 1997).

5.5. CYP2C19

CYP2C19 (S-mephenytoin hydroxylase) metabolizes hexobarbital, propanolol, omeprazole, impramine, and diazepam to varying extends S-mephenytoin (Bertilsson et al., Polymorphic drug oxidation: Relevance to the treatment of psychiatric disorders, CNS Drugs 5, 200-223, 1996). Importantly in tropical countries, CYP2C19 metabolises proguanil to the active antimalarial metabolite cycloguanil (Ward et al., The activation of the biguanide antimalarial prguanil co-segregates with the mephenytoin oxidation polymoprhism—a panel study, Br. J. Clin. Pharmacol. 31, 689-692, 1991). In addition to the CYP2C19* 1 (wildtype) allele, several polymorphisms are known, including by way of non-limiting example CYP2C19*1, CYP2C19*2, CYP2C19*2A (G681A), CYP2C19*3 (G636A), CYP2C19*4, CYP2C19*5A, and CYP2C19*5B.

5.6 CYP2E1

CYP2E 1 is an ethanol inducible enzyme important for the metabolism of ethanol, paracetamol, N-nitrosamines, acrylamide, butadiene, styrene, trichloroethylene, vinyl chloride, and a number of organic solvents (Guengerich et al., Role of human cytochrome P-450 IIE1 in the oxidation of many low molecular weight cancer suspects, Chem. Res. Toxicol. 4, 168-179, 1991). In many cases, this metabolism leads to the formation of more toxic compounds. The interindividual variation in CYP2E1 enzymatic activity may thus affect the individual susceptibility to many chemicals.

The drug chlorzoxazone has been put forward as an in vivo probe for CYP2E1 activity via bioassay. Phenotyping studies with Chlorzoxazone show a 4-5 fold variation in clearance of the drug in humans (Daly, Molecular basis of polymorphic drug metabolism, J. Mol. Med. 73, 539-553, 1995) and a 50-fold variation in the expression of CYP2E1 (Stephens et al., 1994, Pharmacogenetics 4, 185-192). In addition to the CYP2EI *1 (wildtype) allele, polymorphisms are known such as a point mutation in exon 2 of the CYP2E1 gene (CYP2E1 *2), and CYP2E1*5 (G1293C; C1053T). Several polymorphisms in the promotor region ofCYP2E1 have been described (e.g., CYP2E*1D, which has 8 repeats in 5′ region), but it is not clear how, if at all, these variations affect the CYP2E1 phenotype.

5.7 CYP3A

The CYP3A enzyme subfamily is the most abundant of the human cytochrome enzymes. These account for many clinically significant and important interactions; for example, inhibitors of CPY3 A enzymes such as ketoconazole or clarithromycin can cause levels of concomitantly administered cisapride or terfenadine to elevate to cardiotoxic levels. Drugs metabolized include, but are not limited to, Benzodiazepines, Calcium Channel Blockers, Cisapride (Propulsid), Ethinyl estradiol, Lovastatin, Terfenadine, Theophylline, and Protease Inhibitors (Wilkinson, Cytochrome P4503A (CYP3A) metabolism: prediction of in vivo activity in humans, J Pharmacokinet Biopharm 24:475-90, 1996).

5.8 CYP3A4

CYP3A4 is involved in the metabolism of numerous human carcinogens, steroid hormones, and drugs. A variant allele having a mutation located in the 5′-untranslated region of the CYP3A4 gene has been described (U.S. Pat. No. 6,183,963). The frequency of this variant allele is estimated to be 2% in a Caucasian Canadian control population.

5.9 Agents affecting cytochrome P450 metabolism

The following table provides a list of agents that have been reported to be metabolized by, inhibit, or induce specific cytochrome P450 isoforms. The skilled artisan will recognize that individuals exposed to one or more of these agents may be screened for cytochrome P450 polymorphisms according to the present invention, and the information gained used to select drugs and/or dosages for delivery to the individual.

TABLE 4
List of exemplary agents reported as metabolized by particular
cytochrome P450 isoforms (see http://medicine.iupui.edu/flockhart/)

cytochrome P450 1A2
amitriptyline
caffeine
clomipramine
clozapine
cyclobenzaprine (Flexeril ®)
estradiol
fluvoxamine
haloperidol
N-DeMe imipramine
mexiletine
naproxen
ondansetron
phenacetin
acetaminophen
propranolol
riluzole
ropivacaine
tacrine
theophylline
verapamil
R-warfarin
zileuton
zolmitriptan
cytochrome P450 2B6
bupropion
cyclophosphamide
ifosfamide
cytochrome P450 2C19
Proton Pump Inhibitors:
lansoprazole
omeprazole
pantoprazole
E-3810
Anti-epileptics:
diazepam
phenytoin
S-mephenytoin
phenobarbitone
amitriptyline
citalopram
clomipramine
cyclophosphamide
hexobarbital
N-DeME imipramine
indomethacin
R-mephobarbital
moclobemide
nelfinavir
nilutamide
primidone
progesterone
proguanil
propranolol
teniposide
R-warfarin
cytochrome P450 2C9
NSAIDs:
diclofenac
ibuprofen
meloxicam
naproxen
piroxicam
suprofen
Oral Hypoglycemic Agents:
tolbutamide
glipizide
Angiotensin II Blockers:
losartan
irbesartan
amitriptyline
celecoxib
fluoxetine
fluvastatin glyburide
phenytoin
rosiglitazone
tamoxifen
torsemide
S-warfarin
cytochrome P450 2D6
Beta Blockers:
carvedilol
S-metoprolol
propafenone
timolol
Antidepressants:
amitriptyline
clomipramine
desipramine
imipramine
paroxetine
Antipsychotics:
haloperidol
perphenazine
risperidone
thioridazine
alprenolol
amphetamine
bufuralol
chlorpheniramine
chlorpromazine
O-desMe codeine
debrisoquine
dexfenfluramine
dextromethorphan
encainide
flecainide
fluoxetine
fluvoxamine
lidocaine
metoclopramide
methoxyamphetamine
mexiletine
nortriptyline
minaprine
ondansetron
perhexiline
phenacetin
phenformin
propranolol
quanoxan
sparteine
tamoxifen
tramadol
venlafaxine
cytochrome P450 2E1
Anesthetics:
enflurane
halothane
isoflurane
methoxyflurane
sevoflurane
acetaminophen
aniline
benzene
chlorzoxazone
ethanol
N,N-dimethyl formamide
theophylline
cytochrome P450 3A4, 5, 7
Macrolide antibiotics:
clarithromycin
erythromycin (not 3A5)
Anti-arrhythmics:
quinidine (not 3A5)
Benzodiazepines:
alprazolam
diazepam => 3OH
midazolam
triazolam
Immune Modulators:
cyclosporine
tacrolimus (FK506)
HIV Antivirals:
indinavir
nelfinavir
ritonavir
saquinavir
Prokinetic:
cisapride
Antihistamines:
astemizole
chlorpheniramine
terfenidine
Calcium Channel Blockers:
amlodipine
diltiazem
felodipine
lercanidipine
nifedipine
nisoldipine
nitrendipine
verapamil
HMG CoA Reductase Inhibitors:
atorvastatin
cerivastatin
lovastatin
simvastatin
Steroid 6 β-OH:
estradiol
hydrocortisone
progesterone
testosterone
alfentanyl
buspirone
caffeine
cocaine
dapsone
codeine
dextromethorphan
fentanyl
finasteride
haloperidol
lidocaine
methadone
odanestron
pimozide
propranolol
quinine
salmeterol
sildenafil
tamoxifen
taxol
terfenadine
trazodone
vincristine
zaleplon
zolpidem

List of exemplary agents reported as inhibiting particular cytochrome
P450 isoforms (see http://medicine.iupui.edu/flockhart/)

cytochrome P450 1A2
amiodarone
cimetidine
fluoroquinolones
fluvoxamine
furafylline
interferon
methoxsalen
mibefradil
ticlopidine
cytochrome P450 2B6
thiotepa
cytochrome P450 2C19
cimetidine
felbamate
fluoxetine
fluvoxamine
indomethacin
ketoconazole
lansoprazole
modafinil
omeprazole
paroxetine
probenicid
ticlopidine
topiramate
cytochrome P450 2C9
amiodarone
fluconazole
fluvastatin
fluvoxamine
isoniazid
lovastatin
paroxetine
phenylbutazone
probenicid
sertraline
sulfamethoxazole
sulfaphenazole
teniposide
trimethoprim
zafirlukast
cytochrome P450 2D6
amiodarone
celecoxib
chlorpromazine
chlorpheniramine
cimetidine
clomipramine
cocaine
doxorubicin
fluoxetine
halofantrine
haloperidol
levomepromazine
metoclopramide
methadone
mibefradil
moclobemide
paroxetine
quinidine
ranitidine
ritonavir
sertraline
terbinafine
cytochrome P450 2E1
diethyl dithiocarbamate
disulfiram
cytochrome P450 3A4, 5, 7
HIV Antivirals:
delaviridine
indinavir
nelfinavir
ritonavir
saquinavir
amiodarone
cimetidine
ciprofloxacin
clarithromycin
diethyl-dithiocarbamate
diltiazem
erythromycin
fluconazole
fluvoxamine
gestodene
grapefruit juice
itraconazole
ketoconazole
mifepristone
nefazodone
norfloxacin
norfluoxetine
mibefradil
troleandomycin

List of exemplary agents reported as inducing particular cytochrome
P450 isoforms (see http://medicine.iupui.edu/flockhart/)

cytochrome P450 1A2
broccoli
brussel sprouts
char-grilled meat
insulin
methyl cholanthrene
modafinil nafcillin?
beta-naphthoflavone
omeprazole
tobacco
cytochrome P450 2B6
phenobarbital
rifampin
cytochrome P450 2C19
carbamazepine
norethindrone
prednisone
rifampin
cytochrome P450 2C9
rifampin
secobarbital
cytochrome P450 2D6
dexamethasone
rifampin
cytochrome P450 2E1
ethanol
isoniazid
cytochrome P450 3A4, 5, 7
HIV Antivirals:
rifabutin
efavirenz
nevirapine
barbiturates
carbamazepine
glucocorticoids
modafinil
phenobarbital
phenytoin
pioglitazone
rifampin
St. John's wort
troglitazone

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A method of identifying one of a plurality of preselected polymorphisms that may be present in a cytochrome P450 2D6 gene sequence in a sample, the method comprising:
(a) incubating a reaction comprising:
(i) an amount of nucleic acid obtained from said sample sufficient for primer extension, wherein said nucleic acid comprises said P450 2D6 gene sequence,
(ii) a nucleic acid polymerase,
(iii) a plurality of extension primers that specifically bind to a P450 2D6 gene sequence, and that, when extended by one nucleotide at the 3′ end, comprise a nucleotide indicative of one of a plurality of preselected polymorphisms in said P450 2D6 gene sequence, and
(iv) a set of distinctively labeled ddNTPs,
under conditions such that at least one of said extension primers is distinctively labeled by addition of one of said ddNTPs comprising a label to the 5′-end of said detection primer, to generate at least one labeled nucleic acid corresponding to at least one of said preselected polymorphisms; and
(b) relating the labeled nucleic acid to the identity of said polymorphism in said sample.

2. The method of claim 1, wherein said nucleic acid is obtained from said sample by amplification of DNA in said sample.

3. The method of claim 2, wherein said amplification is accomplished by the addition of nucleic acid primers having SEQ ID NOs 1 to 8.

4. The method of claim 1, wherein said relating step (b) comprises mobilizing said labeled nucleic acid(s) by electrophoresis.

5. The method of claim 4, wherein said electrophoresis is capillary electrophoresis.

6. The method claim 1, wherein one or more of steps (a), (b) or (c), or combinations thereof, are automated.

7. The method of claim 1, wherein said distinctive labeled ddNTPs are fluorescently labeled.

8. The method of claim 1, wherein said plurality of preselected cytochrome P450 2D6 polymorphisms are independently selected from the group consisting of a duplication, a deletion, an inversion, an insertion, a translocation, a polymorphism resulting in aberrant RNA splicing, and a single nucleotide polymorphism.

9. The method of claim 1, wherein said preselected cytochrome P450 2D6 polymorphisms are selected from the group consisting of CYP2D6*3, CYP2D6*4, CYP2D6*5, CYP2D6*6, CYP2D6*7, CYP2D6*8, CYP2D6*10, CYP2D6*17 and CYP2D6*N×2.

10. The method of claim 9, wherein said extension primers have sequences selected from the group consisting of SEQ ID NOS: 9 through 19.

11. The method of claim 1, wherein said sample is a human sample.

12. The method of claim 1, wherein said polymorphism is associated with phenotype selected from the group consisting of having a reduced rate or degree of metabolism of one or more xenobiotics or endobiotics, an increased rate or degree of metabolism of one or more xenobiotics or endobiotics, a decreased or increased specificity for one or more xenobiotics or endobiotics, and combinations thereof.

13. The method of claim 12, wherein said xenobiotic is a toxin, a carcinogen or a narcotic, or a metabolic precursor thereof.

14. The method of claim 13, wherein said sample is a sample from a subject having a genetic predisposition to suffer from a toxin, a carcinogen, or a narcotic.

15. The method of claim 12, wherein said xenobiotic is a therapeutic drug or a metabolic precursor thereof.

16. The method of claim 15, wherein said therapeutic drug is a cardioactive drug or a psychoactive drug.

17. The method of claim 15, wherein said subject has a disease or disorder that may be treated by said therapeutic drug.

18. The method of claim 1 further comprising detection of wildtype P450 2D6.

19. A method of identifying a polymorphism in a cytochrome P450 2D6 gene sequence in a sample, the method comprising:
generating from said sample a labeled nucleic acid comprising a means for distinguishing amongst a plurality of preselected polymorphisms in said P450 2D6 gene; and
relating said labeled nucleic acid to the identity of said polymorphism in said sample.

20. The method of claim 19, wherein said nucleic acid is obtained from said sample by amplification of DNA in said sample.

21. The method of claim 20, wherein said amplification is accomplished by the addition of nucleic acid primers having SEQ ID NOs 1 to 8.

22. The method of claim 19, wherein said means for distinguishing amongst a plurality of preselected polymorphisms comprises a primer extension reaction with distinctively labeled ddNTPs and size separation of labeled primers by electrophoresis.

23. The method of claim 22, wherein said electrophoresis is capillary electrophoresis.

24. The method claim 19, wherein said means for distinguishing amongst a plurality of preselected polymorphisms is automated.

25. The method of claim 22, wherein said distinctively labeled ddNTPs are fluorescently labeled.

26. The method of claim 19, wherein said plurality of preselected cytochrome P450 2D6 polymorphisms are independently selected from the group consisting of a duplication, a deletion, an inversion, an insertion, a translocation, a polymorphism resulting in aberrant RNA splicing, and a single nucleotide polymorphism.

27. The method of claim 19, wherein said preselected cytochrome P450 2D6 polymorphisms are selected from the group consisting of CYP2D6*3, CYP2D6*4, CYP2D6*5, CYP2D6*6, CYP2D6*7, CYP2D6*8, CYP2D6*10, CYP2D6*17 and CYP2D6*N×2.

28. The method of claim 27, wherein said extension primers have sequences selected from the group consisting of SEQ ID NOS: 9 through 19.

29. The method of claim 19, wherein said sample is a human sample.

30. A method of selecting a therapeutic drug, or a prodrug thereof, to treat a subject suffering from a disease or disorder, said method comprising:
selecting said therapeutic drug or prodrug to be compatible with a cytochrome P450 2D6 genotype of said subject identified by the method of claim 1 or 19.

31. A method of selecting a dosage of a therapeutic drug, or a prodrug thereof, to treat a subject suffering from a disease or disorder, said method comprising:
selecting said dosage to be compatible with a cytochrome P450 2D6 genotype of said subject identified by the method of claim 1 or 19.

32. The method of claim 31 or 32, wherein said P450 2D6 genotype of said subject comprises a cytochrome P450 2D6 gene selected from the group consisting of CYP2D6*3, CYP2D6*4, CYP2D6*5, CYP2D6*6, CYP2D6*7, CYP2D6*8, CYP2D6*10, CYP2D6*17 and CYP2D6*N×2.

33. A substantially purified nucleic acid that hybridizes to the P450 2D6 gene, said nucleic acid selected from the group consisting of SEQ ID NOs 9 to 19.

34. The substantially purified nucleic acid of claim 33 wherein said nucleic acid is SEQ ID NO:11.

35. The substantially purified nucleic acid of claim 33 wherein said nucleic acid is SEQ ID NO:14.

36. A method of identifying at least one of a preselected polymorphism that may be present in a cytochrome P450 2D6 gene sequence in a human sample, the method comprising:
(a) incubating a reaction comprising:
(i) an amount of nucleic acid obtained from said sample sufficient for primer extension, wherein said nucleic acid comprises said P450 2D6 gene sequence,
(ii) a nucleic acid polymerase,
(iii) at least one extension primer selected from the group consisting of SEQ ID NOs 9 to 19, and
(iv) a set of distinctively labeled ddNTPs,
under conditions such that said at least one extension primer is distinctively labeled by addition of one of said ddNTPs comprising a label to the 5′-end of said at least one detection primer, to generate at least one labeled nucleic acid corresponding to at least one of said preselected polymorphisms; and
(b) relating the labeled nucleic acid to the identity of said polymorphism in said sample.

37. The method of claim 36, wherein said nucleic acid is obtained from said sample by amplification of DNA in said sample.

38. The method of claim 37, wherein said amplification is accomplished by the addition of nucleic acid primers having SEQ ID NOs 1 to 8.

39. The method of claim 36, wherein said relating step (b) comprises mobilizing said labeled nucleic acid(s) by electrophoresis.

40. The method of claim 39, wherein said electrophoresis is capillary electrophoresis.

41. The method claim 36, wherein one or more of steps (a), (b) or (c), or combinations thereof, are automated.

42. The method of claim 36, wherein said distinctive labeled ddNTPs are fluorescently labeled.

43. The method of claim 36, wherein said primers are SEQ ID NO: 17, 18 and 19.

44. The method of claim 36, wherein said primers are SEQ ID NO: 11.

45. The method of claim 36, wherein said primers are SEQ ID NO: 11 ND 14.
